SEQUENCE LISTING (20 sequences)

SEQ ID NO: 1 - protein, 328 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 2 - protein, 1143 aa, Homo sapiens
MYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 3 - protein, 161 aa, Homo sapiens
MNLAISIALLLTVLQVSRGQKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKCEGISLLAQNTSWLLLLLLSLSLLQATDFMSL

SEQ ID NO: 4 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 5 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 6 - protein, 556 aa, Homo sapiens [sequence omitted]

SEQ ID NO: 7 - protein, 322 aa, Homo sapiens
WPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 8 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 9 - protein, 725 aa, Homo sapiens
MSFPKAPLKRFNDPSGCAPSPGAYDVKTLEVLKGPVSFQKSQRFKQQKESKQNLNVDKDTTLPASARKVKSSESKKESQKNDKDLKILEKEIRVLLQERGAQDRRIQDLETELEKMEARLNAALREKTSLSANNATLEKQLIELTRTNELLKSKFSENGNQKNLRILSLELMKLRNKRETKMRGMMAKQEGMEMKLQVTQRSLEESQGKIAQLEGKLVSIEKEKIDEKSETEKLLEYIEEISCASDQVEKYKLDIAQLEENLKEKNDEILSLKQSLEENIVILSKQVEDLNVKCQLLEKEKEDHVNRNREHNENLNAEMQNLKQKFILEQQEREKLQQKELQIDSLLQQEKELSSSLHQKLCSFQEEMVKEKNLFEEELKQTLDELDKLQQKEEQAERLVKQLEEEAKSRAEELKLLEEKLKGKEAELEKSSAAHTQATLLLQEKYDSMVQSLEDVTAQFESYKALTASEIEDLKLENSSLQEKAAKAGKNAEDVQHQILATESSNQEYVRMLLDLQTKSALKETEIKEITVSFLQKITDLQNQLKQQEEDFRKQLEDEEGRKAEKENTTAELTEEINKWRLLYEELYNKTKPFQLQLDAFEVEKQALLNEHGAAQEQLNKIRDSYAKLLGHQNLKQKIKHVVKLKDENSQLKSEVSKLRCQLAKKKQSETKLQEELNKVLGIKHFDPSKAFHHESKENFALKTPLKEGNTNCYRAPMECQESWK

SEQ ID NO: 10 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 11 - DNA, 1158 bp, Homo sapiens
atgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtga

SEQ ID NO: 12 - DNA, 3915 bp, Homo sapiens
atgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcatag

SEQ ID NO: 13 - DNA, 486 bp, Homo sapiens
atgaacctggccatcagcatcgctctcctgctaacagtcttgcaggtctcccgagggcagaaggtgaccagcctaacggcctgcctagtggaccagagccttcgtctggactgccgccatgagaataccagcagttcacccatccagtacgagttcagcctgacccgtgagacaaagaagcacgtgctctttggcactgtgggggtgcctgagcacacataccgctcccgaaccaacttcaccagcaaatacaacatgaaggtcctctacttatccgccttcactagcaaggacgagggcacctacacgtgtgcactccaccactctggccattccccacccatctcctcccagaacgtcacagtgctcagagacaaactggtcaagtgtgagggcatcagcctgctggctcagaacacctcgtggctgctgctgctcctgctctccctctccctcctccaggccacggatttcatgtccctgtga

SEQ ID NO: 14 - DNA, 1137 bp, Homo sapiens
atggtcctcctttggctcacgctgctcctgatcgccctgccctgtctcctgcaaacgaaggaagatccaaacccaccaatcacgaacctaaggatgaaagcaaaggctcagcagttgacctgggaccttaacagaaatgtgaccgatatcgagtgtgttaaagacgccgactattctatgccggcagtgaacaatagctattgccagtttggagcaatttccttatgtgaagtgaccaactacaccgtccgagtggccaacccaccattctccacgtggatcctcttccctgagaacagtgggaagccttgggcaggtgcggagaatctgacctgctggattcatgacgtggatttcttgagctgcagctgggcggtaggcccgggggcccccgcggacgtccagtacgacctgtacttgaacgttgccaacaggcgtcaacagtacgagtgtcttcactacaaaacggatgctcagggaacacgtatcgggtgtcgtttcgatgacatctctcgactctccagcggttctcaaagttcccacatcctggtgcggggcaggagcgcagccttcggtatcccctgcacagataagtttgtcgtcttttcacagattgagatattaactccacccaacatgactgcaaagtgtaataagacacattcctttatgcactggaaaatgagaagtcatttcaatcgcaaatttcgctatgagcttcagatacaaaagagaatgcagcctgtaatcacagaacaggtcagagacagaacctccttccagctactcaatcctggaacgtacacagtacaaataagagcccgggaaagagtgtatgaattcttgagcgcctggagcaccccccagcgcttcgagtgcgaccaggaggagggcgcaaacacacgtgcctggcggacgtcgctgctgatcgcgctggggacgctgctggccctggtctgtgtcttcgtgatctgcagaaggtatctggtgatgcagagactctttccccgcatccctcacatgaaagaccccatcggtgacagcttccaaaacgacaagctggtggtctgggaggcgggcaaagccggcctggaggagtgtctggtgactgaagtacaggtcgtgcagaaaacttga

SEQ ID NO: 15 - DNA, 903 bp, Homo sapiens
atggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctga

SEQ ID NO: 16 - DNA, 1671 bp, Homo sapiens
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtga

SEQ ID NO: 17 - DNA, 972 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 18 - DNA, 1487 bp, Homo sapiens
tttgtagtgggaggatacctccagagaggctgctgctcattgagctgcactcacatgaggatacagactttgtgaagaaggaattggcaacactgaaacctccagaacaaaggctgtcactaaggtcccgctgccttgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggatcaatgaagactaaatataaaaaacattttcttgaatggcatgctagtagcagtgagcaaaggtgtgggtgtgaaaggtttccaaaaaaagttcagcatgaaggatgccatatatgttgttgccaacacttggaacacaatgactaaagacatagttgtgcatgcctggcacaacatcaagcctgtgattgtgtttattgatgatgttgaacaagtggtaactttaaaggattctgtatgccaagtgaaaaaaaaagatgtctgacctccttacatat

SEQ ID NO: 19 - DNA, 3144 bp, Homo sapiens
attctttcttcgtgttcctgtgcgggattggtgtgcccaggggtttggctttccaattggctaacgccggggtgggtggggaatgtggggagatttgaatttgaaaccggtagggagtgataatccgcattcagttgtcgaggagtgccagtcaccttcagtttctggagctggccgtcaacatgtcctttcctaaggcgcccttgaaacgattcaatgacccttctggttgtgcaccatctccaggtgcttatgatgttaaaactttagaagtattgaaaggaccagtatcctttcagaaatcacaaagatttaaacaacaaaaagaatctaaacaaaatcttaatgttgacaaagatactaccttgcctgcttcagctagaaaagttaagtcttcggaatcaaagaaggaatctcaaaagaatgataaagatttgaagatattagagaaagagattcgtgttcttctacaggaacgtggtgcccaggacaggcggatccaggatctggaaactgagttggaaaagatggaagcaaggctaaatgctgcactaagggaaaaaacatctctctctgcaaataatgctacactggaaaaacaacttattgaattgaccaggactaatgaactactaaaatctaagttttctgaaaatggtaaccagaagaatttgagaattctaagcttggagttgatgaaacttagaaacaaaagagaaacaaagatgaggggtatgatggctaagcaagaaggcatggagatgaagctgcaggtcacccaaaggagtctcgaagagtctcaagggaaaatagcccaactggagggaaaacttgtttcaatagagaaagaaaagattgatgaaaaatctgaaacagaaaaactcttggaatacatcgaagaaattagttgtgcttcagatcaagtggaaaaatacaagctagatattgcccagttagaagaaaatttgaaagagaagaatgatgaaattttaagccttaagcagtctcttgaggagaatattgttatattatctaaacaagtagaagatctaaatgtgaaatgtcagctgcttgaaaaagaaaaagaagaccatgtcaacaggaatagagaacacaacgaaaatctaaatgcagagatgcaaaacttaaaacagaagtttattcttgaacaacaggaacgtgaaaagcttcaacaaaaagaattacaaattgattcacttctgcaacaagagaaagaattatcttcgagtcttcatcagaagctctgttcttttcaagaggaaatggttaaagagaagaatctgtttgaggaagaattaaagcaaacactggatgagcttgataaattacagcaaaaggaggaacaagctgaaaggctggtcaagcaattggaagaggaagcaaaatctagagctgaagaattaaaactcctagaagaaaagctgaaagggaaggaggctgaactggagaaaagtagtgctgctcatacccaggccaccctgcttttgcaggaaaagtatgacagtatggtgcaaagccttgaagatgttactgctcaatttgaaagctataaagcgttaacagccagtgagatagaagatcttaagctggagaactcatcattacaggaaaaagcggccaaggctgggaaaaatgcagaggatgttcagcatcagattttggcaactgagagctcaaatcaagaatatgtaaggatgcttctagatctgcagaccaagtcagcactaaaggaaacagaaattaaagaaatcacagtttcttttcttcaaaaaataactgatttgcagaaccaactcaagcaacaggaggaagactttagaaaacagctggaagatgaagaaggaagaaaagctgaaaaagaaaatacaacagcagaattaactgaagaaattaacaagtggcgtctcctctatgaagaactatataataaaacaaaaccttttcagctacaactagatgcttttgaagtagaaaaacaggcattgttgaatgaacatggtgcagctcaggaacagctaaataaaataagagattcatatgctaaattattgggtcatcagaatttgaaacaaaaaatcaagcatgttgtgaagttgaaagatgaaaatagccaactcaaatcggaagtatcaaaactccgctgtcagcttgctaaaaaaaaacaaagtgagacaaaacttcaagaggaattgaataaagttctaggtatcaaacactttgatccttcaaaggcttttcatcatgaaagtaaagaaaattttgccctgaagaccccattaaaagaaggcaatacaaactgttaccgagctcctatggagtgtcaagaatcatggaagtaaacatctgagaaacctgttgaagattatttcattcgtcttgttgttattgatgttgctgttattatatttgacatgggtattttataatgttgtatttaattttaactgccaatccttaaatatgtgaaaggaacattttttaccaaagtgtcttttgacattttattttttcttgcaaatacctcctccctaatgctcacctttatcacctcattctgaaccctttcgctggctttccagcttagaatgcatctcatcaacttaaaagtcagtatcatattattatcctcctgttctgaaaccttagtttcaagagtctaaaccccagattcttcagcttgatcctggaggtcttttctagtctgagcttctttagctaggctaaaacaccttggcttgttattgcctctactttgattctgataatgctcacttggtcctacctattatccttctacttgtccagttcaaataagaaataaggacaagcctaacttcatagaaacctctctatttttaatcagttgtttaataatttacaggttcttaggctccatcctgtttgtatgaaattataatctgtggattggcctttaagcctgcattcttaacaaactcttcagttaattcttagatacactaaaaatctgagaaactctacatgtaactatttcttcagagtttgtcatatactgcttgtcatctgcatgtctactcagcatttgattaacatttgtgtaatatgaaataaaattacacagtaagtcatttaaccaattaaaaa

SEQ ID NO: 20 - DNA, 2784 bp, Homo sapiens
ggaaggcttgcacagggtgaaagctttgcttctctgctgctgtaacagggactagcacagacacacggatgagtggggtcatttccagatattaggtcacagcagaagcagccaaaatggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaattctaagctggtgccccactgaattttgtgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2013/0337474 A1
(43) Pub. Date: Dec. 19, 2013

(54) DETECTION OF ACUTE MYELOID LEUKAEMIA

(71) Applicants: Paresh Vyas, Oxford (GB); Nicolas Goardon, Oxford (GB); Sylvie Freeman, Edgbaston (GB)

(72) Inventors: Paresh Vyas, Oxford (GB); Nicolas Goardon, Oxford (GB); Sylvie Freeman, Edgbaston (GB)

(73) Assignee: ISIS INNOVATION LIMITED, Oxford (GB)

(21) Appl. No.: 13/995,347

(22) PCT Filed: Dec. 21, 2011
(86) PCT No.: PCT/GB11/52549

§ 371 Date: Sep. 3, 2013

(30) Foreign Application Priority Data
Dec. 21, 2010 (GB) 10211623.2

(51) Int. Cl. G01N 33/50

(52) U.S. Cl. CPC G01N 33/5011
USPC 435/7.23; 530/389.7

(57) ABSTRACT

The present invention relates to diagnostic screens, antibodies, methods and kits for detection/prognosis of acute myeloid leukaemia. The diagnostic screen detects the presence (+) or absence (−) of the cell surface polypeptide markers i) CD34+; ii) CD45RA+; and iii) CD90− and/or CD123+. Antibodies specific for one or more of said cell surface polypeptide markers may be used in the diagnostic screen of the invention. Diagnostic and prognostic methods for detecting and monitoring minimal residual disease based on said screen also form part of the invention.

The present invention relates to diagnostic markers of acute myeloid leukaemia, to a diagnostic screen based on said markers, and to the use of said screen in diagnostic and prognostic methods.

Human Acute Myeloid Leukaemia (AML) is an aggressive cancer of white blood cells and is the most common adult acute leukaemia. In more detail, AML is a cancer of the myeloid line of blood cells. It is characterized by the rapid growth of an abnormal white blood cell population. Approximately 80% of AML patients are over the age of 60 and the overall survival of this patient group lies at only approximately 5%.

AML can be classified into several subgroups. By way of example, classification according to the World Health Organization (WHO) criteria is based on examination of bone marrow aspirate or a blood sample via light microscopy. Alternatively, bone marrow or blood may be tested for chromosomal translocations by routine cytogenetic methods or fluorescent in situ hybridisation (FISH), and for specific genetic mutations (such as mutations in the FLT3, NPM1 and CEBPA genes) may be detected by polymerase chain reaction (PCR). Immunophenotyping is another method that may be used to identify the AML subtype, which involves detection of cell surface and cytoplasmic markers using flow cytometry.

Flow cytometry is a technique for counting and examining microscopic particles such as cells by suspending them in a stream of fluid and capturing the light that emerges from each cell as it passes through a laser beam. Cell surface molecules often referred to as “cluster of differentiation” (CD) molecules may be exploited in flow cytometry to characterise cell populations. For example, in fluorescence-activated cell sorting, a diagnostic antibody (labelled with a fluorophore) is employed, which binds to a surface molecule (e.g. a CD molecule) present on and characteristic of the cell population in question. Thereafter, the flourophore (attached to the antibody) is activated by a laser beam and the fluorescence signal detected by the flow cytometer. In this manner, fluorescently-labelled antibodies can be used to detect and sort cells displaying a specific CD molecule (or set of CD molecules).

Current AML therapies typically involve induction chemotherapy followed by post-induction therapy. The goal of induction chemotherapy is to reduce the amount of leukaemic cells to less than 5% of all the nucleated cells in a bone marrow sample. Regrettably, this level of reduction of leukaemic cells is not enough to prevent disease recurrence (i.e. relapse) and almost all patients relapse without post-induction therapy. Post-induction therapy typically involves further cycles of chemotherapy, and in some cases, a hematopoietic stem cell transplant that aims to eliminate minimal residual disease (MRD). MRD is the population of leukaemic cells that is recaltricant to therapy. It is thought that this population of cells contains a sub-population of cells termed a leukaemic stem cell (LSC) population that is largely quiescent and serves to sustain disease.

Current methods used to detect MRD include real time quantitative PCR (RQ-PCR) or by multi-parameter flow cytometry (MFC). However, RQ-PCR based MRD assessment is not possible in approximately half of patients with AML. In addition, and despite recent technical developments, there is still a lack of a validated MFC methodology demonstrating clinical utility—current sensitivity levels of MFC are at least 1 log below real time that of RQ-PCR assays.

There is, therefore, a need to provide an alternative and/or improved diagnostic screen for acute myeloid leukaemia. In addition, there is a need to provide an alternative and/or improved method for diagnosis and/or prognosis of acute myeloid leukaemia. In particular, there is a need to provide an alternative and/or improved method to detect and monitor MRD for acute myeloid leukaemia.

The present invention solves one or more of the above mentioned problems.

In one aspect, the invention provides a diagnostic screen for detecting acute myeloid leukaemia, wherein said screen detects the presence (+) or absence (−), as indicated below, of the following cell surface polypeptide markers:
i) CD34⁺;
ii) CD45RA⁺; and
iii) CD90⁻ and/or CD123⁺.

A cell surface polypeptide marker may be displayed (at least in part) on the extracellular surface of a cell. Markers of the present invention may include CD34, CD45RA, CD90, CD123, CD38, CD19, CD47, CCR8, RHAMM and/or CD86. CD34 is a heavily glycosylated, 105-120 kDa transmembrane glycoprotein expressed on hematopoietic progenitor cells, vascular endothelial cells and some fibroblasts. The CD34 cytoplasmic domain is a target for phosphorylation by activated protein kinase C, suggesting a role for CD34 in signal transduction. CD34 may also play a role in adhesion of certain antigens to endothelium. CD45R, also designated CD45 and PTPRC, has been identified as a transmembrane glycoprotein, broadly expressed among hematopoietic cells. Multiple isoforms of CD45R are distributed throughout the immune system according to cell type including CD45RA. CD45R functions as a phosphotyrosine phosphatase, a vital component for efficient tyrosine phosphorylation induction by the TCR/CD3 complex. CD90 is a 25-37 kDa heavily N-glycosylated, glycophosphatidylinositol (GPI) anchored conserved cell surface protein originally discovered as a thymocyte antigen. The CD123 antigen (also known as interleukin-3 receptor) is a molecule found on cells which helps transmit the signal of interleukin-3, a soluble cytokine important in the immune system. CD38, also known as cyclic ADP ribose hydrolase is a glycoprotein found on the surface of many immune cells (white blood cells. CD38 is thought to function in cell adhesion, signal transduction and calcium signaling. CD19 is a 95 kDa type-I transmembrane glycoprotein that belongs to the immunglobulin superfamily. It is expressed on B cells throughout most stages of B cell differentiation and associates with CD21, CD81, and CD225 (Leu-13) forming a signal transduction complex. CD19 functions as a regulator in B cell development, activation, and differentiation. CD47 is an integral membrane protein that plays a role in the regulation of cation fluxes across cell membranes. It is also a receptor for the C-terminal cell binding domain of thrombospondin (SIRP). CD47 is expressed on hemopoietic cells, epithelial cells, endothelial cells, fibroblasts, brain and mesenchymal cells. Chemokine receptor 8, also known as CCR8 is a member of the beta chemokine receptor family CCR8. HMMR hyaluronan-mediated motility receptor (RHAMM) is a cell surface receptor. The CD86 gene encodes a type I membrane protein that is expressed on antigen-presenting cells.

The present inventors have unexpectedly found that a combination of the above-mentioned cell surface markers represents a robust diagnostic screen for acute myeloid leukaemia. Diagnostic capacity in this context may also embrace prognostic capacity and diagnosis/monitoring of MRD.

A screen as defined above has many useful applications including diagnostic and prognostic applications such as in clinical guidance and for determining therapy, for patient management and for assessing treatment efficacy.

In one embodiment, the invention provides a diagnostic screen as defined above, wherein the marker iii) is CD90⁻.

In another embodiment, the invention provides a diagnostic screen as defined above, wherein the marker iii) is CD123⁺.

In another embodiment, the invention provides a diagnostic screen as defined above, wherein the marker iii) is CD90⁻ and CD123⁺.

In a further embodiment, the invention provides a diagnostic screen as defined above, further comprising the cell surface polypeptide marker CD38+. In an alternative embodiment, the invention provides a diagnostic screen as defined above, further comprising the cell surface polypeptide marker CD38⁻ (i.e. CD38⁻ instead of CD38⁺).

In one embodiment, the invention provides a diagnostic screen as defined above, further comprising one or more (or two or more, or three or more, or four or more) of the cell surface polypeptide markers selected from CD19⁻, CD47^{+/-}, CCR8^{+/-}, CD86⁻ and/or RHAMM^{+/-}. In one embodiment, the invention provides a diagnostic screen as defined above, comprising the cell surface polypeptide marker CD47+. In an alternative embodiment, the invention provides a diagnostic screen as defined above, comprising the cell surface polypeptide marker CD47⁻ (i.e. CD47⁻ instead of CD47⁺). In one embodiment, the invention provides a diagnostic screen as defined above, comprising the cell surface polypeptide marker CCR8+. In an alternative embodiment, the invention provides a diagnostic screen as defined above, comprising the cell surface polypeptide marker CCR8⁻ (i.e. CCR8⁻ instead of CCR8+). In one embodiment, the invention provides a diagnostic screen as defined above, comprising the cell surface polypeptide marker RHAMM+. In an alternative embodiment, the invention provides a diagnostic screen as defined above, comprising the cell surface polypeptide marker RHAMM⁻ (i.e. RHAMM⁻ instead of RHAMM⁺).

In one embodiment, the diagnostic screen comprises one or more antibodies that bind to one or more of the identified markers. Thus, said one or more antibodies may be used to confirm the presence (+) or absence (−) of said cell surface polypeptide markers. In one embodiment, the presence (+) of a marker refers to an elevation in the levels of marker in a sample above a background level. Likewise, the absence (−) of a marker refers to a reduction in the levels of a marker in a sample below a background level. In one embodiment, the elevation in the levels of marker in a sample above a background level is 1 or more (such as 2, 3, 4, 5, 6, 7, 8, 10, 15, 20, 25) flourescence units. In one embodiment a reduction in the levels of a merker in a sample below a background level is 1 or more (such as 2, 3, 4, 5, 6, 7, 8, 10, 15, 20, 25) flourescence units. In this regard, it would be routine for a skilled person in the art to determine the background level of marker expression in a sample. Thus, in one embodiment, said cell surface polypeptide markers may be detected by specific binding of said one or more antibodies.

In one embodiment, the screen comprises one or more antibodies that bind to one or more cell surface polypeptide markers selected from CD34, CD45RA, CD90, CD123, CD38, CD19, CD47, CCR8, CD86 and/or RHAMM.

In one embodiment, the screen comprises a first antibody that binds to CD34, a second antibody that binds to CD45RA, and a third antibody that binds to CD90 and/or to CD123. In another embodiment, the screen comprises a first antibody that binds to CD34, a second antibody that binds to CD45RA, a third antibody that binds to CD90 and/or to CD123, and a fourth antibody that binds to CD38.

Any one or more of said antibodies may bind to one of said markers and not (substantially) to any of the other markers. For example, each of the employed antibodies may bind to one of said markers and not (substantially) to any of the other markers. Alternatively, any one or more of said antibodies may bind to two, three, four, five, six, seven, eight, nine or all ten of said markers.

In one embodiment, the screen comprises three antibodies, wherein:
a first antibody binds to CD34 and preferably not to CD45, CD90 and/or CD123;
a second antibody that binds to CD45RA and preferably not to CD34 CD90 and/or CD123; and
a third antibody that binds to CD90 and preferably not to CD34, CD45RA and/or CD123.

In an alternative embodiment, the screen comprises three antibodies, wherein:
a first antibody binds to CD34 and preferably not to CD45, CD90 and/or CD123;
a second antibody that binds to CD45RA and preferably not to CD34 CD90 and/or CD123; and
a third antibody that binds to CD123 and preferably not to CD34, CD45RA and/or CD90.

In one embodiment, the antibodies of the present invention recognise and bind to specific epitopes of the above mentioned cell surface polypeptide markers. For example, an antibody of the present invention may bind to an epitope in the N-terminal/C-terminal/mid-region domains/extracellular domains of CD34/CD45RA/CD90/CD123/CD38/CD19/CD47/CD86/CCR8 and/or RHAMM. The sequence of CD34, CD45RA, CD90, CD123, CD38, CD19, CD47, CD86/CCR8 and RHAMM are available from the NCBI website (http://www.ncbi.nlm.nih.gov/projects/genome/assembly/grc/human/index.shtml). These protein sequences are provided as SEQ ID NOs: 1-10. The corresponding nucleic acid sequences are provided as SEQ ID NOs: 11-20.

In one embodiment, the antibodies of the present invention may bind to a CD34/CD45RA/CD90/CD123/CD38/CD19/CD47/CCR8, CD86 and/or RHAMM molecules comprising an amino acid sequence having at least 80% (such at least 85%, 90%, 95%, 98%, 99% or 100%) sequence identity to SEQ ID NOs: 1-10, or a fragment thereof.

Conventional methods for determining nucleic acid sequence identity are discussed in more detail later in the specification.

In one embodiment, the antibodies are polyclonal and/or monoclonal antibodies.

In one embodiment, an antibody that binds to one of the above-mentioned cell surface polypeptide markers is one capable of binding that marker with sufficient affinity such that the antibody is useful as a diagnostic/and or prognostic agent. In one embodiment, the term ‘binds’ is equivalent to ‘specifically binds’. An antibody that binds/specifically binds to a cell surface polypeptide marker of interest is one that binds to one of the above mentioned markers with an affinity (Ka) of at least 10⁴ M.

Suitable antibodies of the present invention may include FITC or PE-Cy7 conjugated anti-CD38, PE or FITC-conjugated anti-CD45RA, PE-Cy7-conjugated or APC conjugated anti-CD123, biotin-conjugated anti-CD90, PE-Cy5 or PERCP-conjugated anti-CD34, PE-conjugated CD47, CD19 Horizon V450 and APC-Alexa Fluor 750 or APC-eFluor 780 conjugated streptavidin which are available from a number of different commercial suppliers including BD Biosciences Europe ebioscience, Beckman Coulter and Pharmingen.

In a preferred embodiment, the antibody is a labelled antibody, such as a fluorescently labelled antibody. Suitable labelled compounds include conventionally known labelled compounds, such as fluorescent substances such as cyanine dyes Cy3 (registered trademark of Amersham Life Science), fluorescein isothiacyanate (FITC), allophycocyanin (APC), rhodamine, Phycoerythrin (PE), PE-Cy5 (Phycoerythrin-Cy5), PE-Cy7 (Phycoerythrin-Cy7), APC-Alexa Fluor 750, APC-eFluor 780, Pacific Blue, Horizon V450 and quantum dot, biotin-conjugated; light scattering substances such as gold particles; photo-absorptive substances such as ferrite; radioactive substances such as <125> I; and enzymes such as peroxidase or alkali phosphatase.

In one embodiment of the invention, different antibodies are labelled respectively with mutually distinguishable labels. Labelling may be conducted by binding a labelled compound directly to each antibody. Preferably, the antibodies are labelled with different fluorescent dyes with different fluorescence wavelengths to enable easy discrimination from one another. For example a first antibody may be labelled in red (for example PE-Cy5), a second antibody in orange (for example PI, APC, R-PE) and a third antibody in green (for example Alexa488, FITC). Suitable labelling strategies are routine and known to a person skilled in the art. By way of example, the Lightening Link™ antibody labeling kit may be used (Innova Biosciences, UK).

Methods suitable for detection of the cell surface polypeptide markers of the present invention using labelled antibodies are conventional techniques known to those skilled in the art. For example, when a fluorescent label is used, an antibody that specifically binds to a marker may be detected by observing the emitted fluorescence colour under a microscope. A fluorescent label can also be detected by irradiating a sample with an exciting light—if the label is present, fluorescence is emitted from the sample. Thus, whether a cell is positive or negative for a particular cell surface marker may be judged by using a labelled antibody specific for said marker and observing the emitted fluorescence colour under a microscope. In a preferred embodiment of the invention, fluorescence-activated cell sorting (FACS) is used for detection of the cell surface polypeptide markers/labeled antibodies of the present invention.

In one aspect, the present invention provides a screen (as defined above) for use in a method of diagnosis of acute myeloid leukaemia.

In a related aspect, the invention provides a method for diagnosing acute myeloid leukaemia, said method comprising:
i) contacting an isolated sample containing a blood cell population with a screen that identifies a blood cell having a cell surface phenotype comprising:
   a) CD34⁺;
   b) CD45RA⁺; and
   c) CD90⁻ and/or CD123⁺;
ii) confirming the presence of a blood cell that has a cell surface phenotype comprising:
   a) CD34⁺;
   b) CD45RA⁺; and
   c) CD90⁻ and/or CD123⁺.

In one embodiment, the method of diagnosis comprises:
i) contacting an isolated sample containing a blood cell population with one or more labelled antibodies that bind to
   a) CD34;
   b) CD45RA; and
   c) CD90 and/or CD123;
ii) detecting the presence or absence of said one or more labelled antibodies bound to a blood cell; and
iii) confirming the presence of a blood cell having a cell surface phenotype comprising:
   a) CD34⁺;
   b) CD45RA⁺; and
   c) CD90⁻ and/or CD123⁺.

All embodiments described above for the diagnostic screen apply equally to the method of diagnosis aspect. By way of example, the latter aspect may further comprise identification of the cell surface polypeptide marker CD38+. Alternatively, the latter aspect may further comprise the cell surface polypeptide marker CD38⁻ (i.e. CD38⁻ instead of CD38⁺).

In another aspect, the present invention provides a screen (as defined above) for use in a method of prognosis of acute myeloid leukaemia.

In a related aspect, the invention provides a method for prognosis of acute myeloid leukaemia, said method comprising:
i) contacting an isolated sample containing a blood cell population with a screen that identifies a blood cell having a cell surface phenotype comprising:
   a) CD34⁺;
   b) CD45RA⁺; and
   c) CD90⁻ and/or CD123⁺;
ii) confirming the presence of a blood cell that has a cell surface phenotype comprising:
   a) CD34⁺;
   b) CD45RA⁺; and
   c) CD90⁻ and/or CD123⁺.

In one embodiment, the method of prognosis comprises:
i) contacting an isolated sample containing a blood cell population with one or more labelled antibodies that bind to:
   a) CD34;
   b) CD45RA; and
   c) CD90 and/or CD123;
ii) detecting the presence or absence of said one or more labelled antibodies bound to a blood cell; and
iii) confirming the presence of a blood cell having a cell surface phenotype comprising:
   a) CD34⁺;
   b) CD45RA⁺; and
   c) CD90⁻ and/or CD123⁺.

All embodiments described above for the diagnostic screen apply equally to the method of prognosis aspect. By way of example, the latter aspect may further comprise identification of the cell surface polypeptide marker CD38+. Alternatively, the latter aspect may further comprise the cell surface polypeptide marker CD38⁻ (i.e. CD38⁻ instead of CD38⁺).

In another aspect, the present invention provides a screen (as defined above) for use in a method of identifying a therapeutic candidate for the treatment of acute myeloid leukaemia.

In a related aspect, the invention provides a method of identifying a therapeutic candidate for the treatment of acute myeloid leukaemia, said method comprising:
i) contacting the therapeutic candidate with an isolated sample containing a population of blood cells, wherein said blood cell has a cell surface phenotype comprising:
   a) CD34⁺;
   b) CD45RA⁺; and
   c) CD90⁻ and/or CD123⁺;
ii) incubating said therapeutic candidate with said isolated sample;
iii) contacting said isolated sample after step ii) with a screen that identifies a blood cell having a cell surface phenotype comprising:
   a) CD34⁺;
   b) CD45RA⁺; and
   c) CD90⁻ and/or CD123⁺;
iv) identifying blood cells by step iii) that have a cell surface phenotype comprising:
   a) CD34⁺;
   b) CD45RA⁺; and
   c) CD90⁻ and/or CD123⁺;
v) correlating the number of blood cells identified by step iv) with the number of blood cells present in an isolated sample prior to step i) that have a cell surface phenotype comprising:
   a) CD34⁺;
   b) CD45RA⁺; and
   c) CD90⁻ and/or CD123⁺;
vi) confirming the presence of a therapeutic candidate having anti-acute myeloid leukaemia cell activity by identifying a relative decrease in the number of blood cells in step v) after contact with the therapeutic candidate; or
   confirming the absence of a therapeutic candidate having anti-acute myeloid leukaemia cell activity by identifying no significant relative decrease in the number of blood cells in step v) after contact with the therapeutic candidate.

In one embodiment, the method of identifying a therapeutic candidate for the treatment of acute myeloid leukaemia comprises:
i) contacting the therapeutic candidate with an isolated sample containing a population of blood cells, wherein said blood cell has a cell surface phenotype comprising:
   a) CD34⁺;
   b) CD45RA⁺; and
   c) CD90⁻ and/or CD123⁺;
ii) incubating said therapeutic candidate with said isolated sample;
iii) contacting said isolated sample after step ii) with one or more labelled antibodies that bind to:
   a) CD34;
   b) CD45RA; and
   c) CD90 and/or CD123;
iv) identifying blood cells by step iii) that have a cell surface phenotype comprising:
   a) CD34⁺;
   b) CD45RA⁺; and
   c) CD90⁻ and/or CD123⁺;
v) correlating the number of blood cells identified by step iv) with the number of blood cells present in an isolated sample prior to step i) that have a cell surface phenotype comprising:
   a) CD34⁺;
   b) CD45RA⁺; and
   c) CD90⁻ and/or CD123⁺;
vi) confirming the presence of a therapeutic candidate having anti-acute myeloid leukaemia cell activity by identifying a relative decrease in the number of blood cells in step v) after contact with the therapeutic candidate; or
   confirming the absence of a therapeutic candidate having anti-acute myeloid leukaemia cell activity by identifying no significant relative decrease in the number of blood cells in step v) after contact with the therapeutic candidate.

All embodiments described above for the diagnostic screen apply equally to the method of identifying a therapeutic candidate aspect. By way of example, the latter aspect may further comprise identification of the cell surface polypeptide marker CD38+. Alternatively, the latter aspect may further comprise the cell surface polypeptide marker CD38⁻ (i.e. CD38⁻ instead of CD38⁺).

In another aspect, the present invention provides a screen (as defined above) for use in a method of monitoring efficacy of a therapeutic molecule in treating acute myeloid leukaemia.

In a related aspect, the invention provides a method for monitoring efficacy of a therapeutic molecule in treating acute myeloid leukaemia, said method comprising:
i) contacting an isolated sample from a patient, wherein said patient has been administered the therapeutic molecule, with a screen that identifies a blood cell having a cell surface phenotype comprising:
   a) CD34⁺;
   b) CD45RA⁺; and
   c) CD90⁻ and/or CD123⁺;
ii) identifying blood cells by step i) that have a cell surface phenotype comprising:
   a) CD34⁺;
   b) CD45RA⁺; and
   c) CD90⁻ and/or CD123⁺;
iii) correlating the number of blood cells identified by step ii) with the number of blood cells present in an isolated sample taken from a patient prior to administration of the therapeutic molecule, wherein said blood cells taken prior to administration of the therapeutic molecule have a cell surface phenotype comprising:
   a) CD34⁺;
   b) CD45RA⁺; and
   c) CD90⁻ and/or CD123⁺;
iv) confirming efficacy of the therapeutic molecule by identifying a relative decrease in the number of blood cells in step iii) after contact with the therapeutic molecule; or
   confirming the absence of efficacy of the therapeutic molecule by identifying a no significant relative decrease in the number of blood cells in step iii) after contact with the therapeutic molecule.

In one embodiment, the invention provides a method for monitoring efficacy of a therapeutic molecule in treating acute myeloid leukaemia, said method comprising:
i) contacting an isolated sample from a patient, wherein said patient has been administered the therapeutic molecule, with a screen that comprises one or more labelled antibodies that bind to:
   a) CD34;
   b) CD45RA; and
   c) CD90 and/or CD123;
ii) identifying blood cells by step i) that have a cell surface phenotype comprising:
   a) CD34⁺;
   b) CD45RA⁺; and
   c) CD90⁻ and/or CD123⁺;
iii) correlating the number of blood cells identified by step ii) with the number of blood cells present in an isolated sample taken from a patient prior to administration of the therapeutic molecule, wherein said blood cells taken prior to administration of the therapeutic molecule have a cell surface phenotype comprising:
   a) CD34⁺;
   b) CD45RA⁺; and
   c) CD90⁻ and/or CD123⁺;
iv) confirming efficacy of the therapeutic molecule by identifying a relative decrease in the number of blood cells in step iii) after contact with the therapeutic molecule; or
   confirming the absence of efficacy of the therapeutic molecule by identifying a no significant relative decrease in the number of blood cells in step iii) after contact with the therapeutic molecule.

All embodiments described above for the diagnostic screen apply equally to the method for monitoring efficacy of a therapeutic molecule in treating acute myeloid leukaemia aspect. By way of example, the latter aspect may further comprise identification of the cell surface polypeptide marker CD38+. Alternatively, the latter aspect may further comprise the cell surface polypeptide marker CD38⁻ (i.e. CD38⁻ instead of CD38⁺).

In one aspect, the invention provides a kit for diagnosis and/or prognosis of acute myeloid leukaemia, said kit comprising at least one antibody that binds to a cell surface polypeptide marker selected from:
i) CD34;
ii) CD45RA; and
iii) CD90 and/or CD123.

In one embodiment, said kit comprises a first antibody that binds to CD34, a second antibody that binds to CD45, and a third antibody that binds to CD90 and/or CD123. In one embodiment, each of said antibodies is different. In another embodiment, each of said antibodies does not substantially bind to any other marker of the present invention—for example: the first antibody does not substantially bind to any of CD45RA, CD90, or CD123; the second antibody does not substantially bind to any of CD34, CD90, or CD123; and the third antibody substantially binds only to one of CD90 or CD123, wherein the third antibody does not substantially bind to either of CD34 or CD45RA. The third antibody may be present that binds to CD90 and not substantially to any of CD34, CD45RA or CD123. A fourth antibody may be present that binds to CD123 and not substantially to any of CD34, CD45RA, or CD90.

In one embodiment, the kit may further comprise instructions explaining how to use the antibodies thereof in a diagnostic/prognostic method of the invention.

All embodiments described above for the diagnostic screen apply equally to the kit aspect. By way of example, the latter aspect may further comprise an antibody that binds to the cell surface polypeptide marker CD38. Thus, in one embodiment, said antibody may constitute a fifth antibody of the kit. In one embodiment, said fifth antibody does not substantially to any other (aforementioned) marker of the invention.

A kit of the present invention may optionally comprise suitable labels as described above (e.g. a fluorophore label) in addition to the one or more antibodies. The kit may optionally contain an instruction manual instructing the user to perform the methods of the present invention.

### DEFINITIONS

In one embodiment, acute myeloid leukaemia includes all AML samples that contain the CD34 cell surface marker.

In one embodiment, the term ‘diagnosis’ is used to mean determining the incidence of AML by examining whether one or more of the cell surface polypeptide markers of the diagnostic screen is present. In one embodiment, diagnosis of AML embraces diagnosis of minimal residual disease (MRD). Accordingly, in one embodiment, reference herein to acute myeloid leukaemia (AML) embraces MRD.

In one embodiment, a sample is obtained from a mammal, such as a human. A suitable sample is a bone marrow or blood sample. The white blood cell population of the sample is preferably extracted or enriched prior to detection of the marker-set with antibodies of the present invention. Methods suitable for extraction of enrichment of the white blood cells from a sample are conventional techniques known to those skilled in the art. By way of example, one approach is to deplete a sample of red cells by red cell lysis. Another approach is to isolate a mononuclear by density centrifugation using a density media like Ficoll. CD34+ cells can be then be purified from mononuclear cells by incubation with magnetic beads coated with CD34 antibody and separating CD34+ cells using a magnet.

In one embodiment, the methods referred to herein are performed in vitro. In one embodiment, the methods referred to herein are performed ex vivo.

The term “antibody” is used in the broadest sense and specifically covers monoclonal and polyclonal antibodies (and fragments thereof) so long as they exhibit the desired biological activity. In particular, an antibody is a protein including at least one or two, heavy (H) chain variable regions (abbreviated herein as VHC), and at least one or two light (L) chain variable regions (abbreviated herein as VLC). The VHC and VLC regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. Sequences of Proteins of Immunological Interest, Fifth Edition, U.S. Department of Health and Human Services, NIH Publication No. 91-3242, 1991, and Chothia, C. et al, J. Mol. Biol. 196:901-917, 1987). Preferably, each VHC and VLC is composed of three CDRs and four FRs, arranged from amino-terminus to carboxy-terminus in the following order: FRI, CDR1, FR2, DR2, FR3, CDR3, FR4. The VHC or VLC chain of the antibody can further include all or part of a heavy or light chain constant region. In one embodiment, the antibody is a tetramer of two heavy immunoglobulin chains and two light immunoglobulin chains, wherein the heavy and light immunoglobulin chains are interconnected by, e.g., disulfide bonds. The heavy chain constant region includes three domains, CH1, CH2 and CH3. The light chain constant region is comprised of one domain, CL. The variable region of the heavy and light chains contains a binding domain that interacts with an antigen. The term “antibody” includes intact immunoglobulins of types IgA, IgG, IgE, IgD, IgM (as well as subtypes thereof), wherein the light chains of the immunoglobulin may be of types kappa or lambda. The term antibody, as used herein, also refers to a portion of an antibody that binds to one of the above-mentioned markers, e.g., a molecule in which one or more immunoglobulin chains is not full length, but which binds to a marker. Examples of binding portions encompassed within the term antibody include (i) a Fab fragment, a monovalent fragment consisting of the VLC, VHC, CL and CHI domains; (ii) a F(ab′)2 fragment, a bivalent fragment comprising two Fab fragments linked by a disulfide bridge at the hinge region; (iii) a Fc fragment consisting of the VHC and CHI domains; (iv) a Fv fragment consisting of the VLC and VHC domains of a single arm of an antibody, (v) a dAb fragment (Ward et al, Nature 341:544-546, 1989), which consists of a VHC domain; and (vi) an isolated complementarity determining region (CDR) having sufficient framework to bind, e.g. an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al. (1988) Science IAI-ATi-Alβ; and Huston et al. (1988) Proc. Natl. Acad. ScL USA 85:5879-5883). Such single chain antibodies are also encompassed within the term antibody. These may be obtained using conventional techniques known to those skilled in the art, and the portions are screened for utility in the same manner as are intact antibodies.

### Antibody Preparation

The antibodies of the present invention can be obtained using conventional techniques known to persons skilled in the art and their utility confirmed by conventional binding studies. By way of example, a simple binding assay is to incubate the cell expressing an antigen with the antibody. If the antibody is tagged with a fluorophore, the binding of the antibody to the antigen can be detected by FACS analysis.

Antibodies of the present invention can be raised in various animals including mice, rats, rabbits, goats, sheep, monkeys or horses. Blood isolated from these animals contains polyclonal antibodies—multiple antibodies that bind to the same antigen. Antigens may also be injected into chickens for generation of polyclonal antibodies in egg yolk. To obtain a monoclonal antibody that is specific for a single epitope of an antigen, antibody-secreting lymphocytes are isolated from an animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies. Methods for producing monoclonal antibodies are conventional techniques known to those skilled in the art (see e.g. Making and Using Antibodies: A Practical Handbook. GC Howard. CRC Books. 2006. ISBN 0849335280). Polyclonal and monoclonal antibodies are often purified using Protein A/G or antigen-affinity chromatography.

### Sequence Homology:

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004). Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

Alignment scores for determining sequence identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ \begin{matrix}\begin{matrix}length of the longer sequence \\ plus the number of gaps introduced\end{matrix} \\ into the longer sequence in \\ order to align the two sequences\end{matrix} ]} \times 100$$

The present invention will now be described, by way of example only, with reference to the accompanying Examples and Figures, in which:

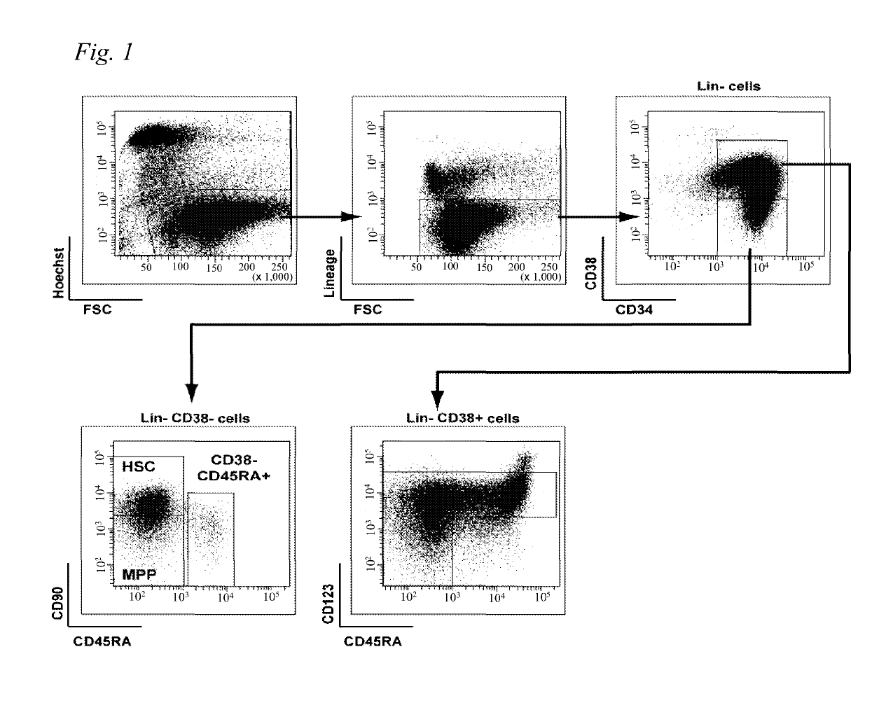

FIG. 1 shows the gating strategy for immunophenotyping and FACS-sorting. stem/progenitor compartments. Normal CD34+ haemopoietic progenitors were analyzed for expression of lineage markers, CD34, CD38, CD45RA, CD90, and CD123. HSCs: Lin-CD34+ CD38− CD90+ CD45RA+: MPPs: Lin-CD34+ CD38− CD90− CD45RA; CD38− CD45RA+: Lin-CD34+ CD38− CD90− CD45RA+; CMPs: Lin-CD34+ CD38+ CD123low/+ CD45RA−, GMPs: Lin-CD34+ CD38+ CD123+ CD45RA+ and MEPs: Lin-CD34+ CD38+ CD123−/low CD45RA−.

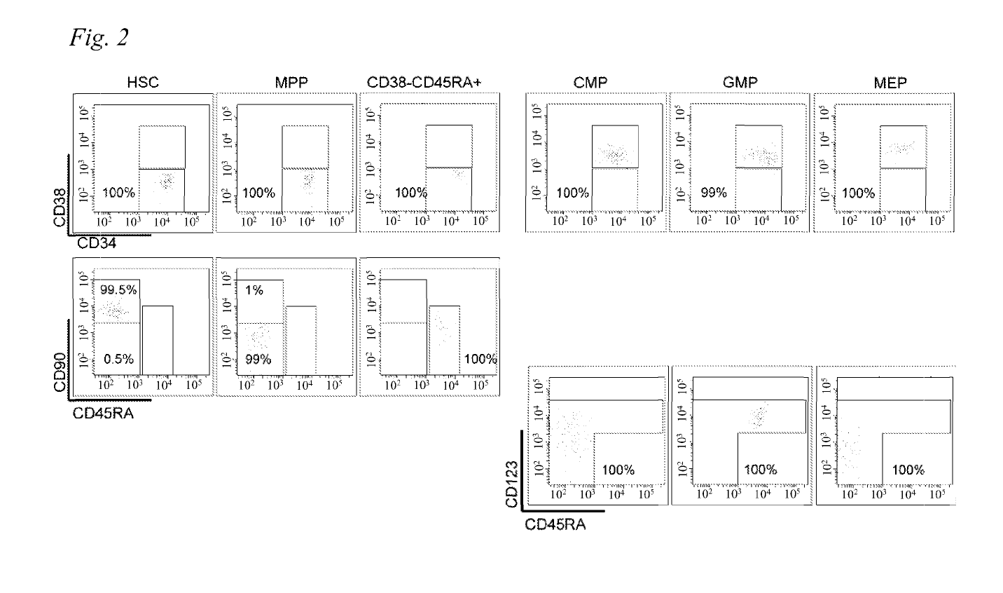

FIG. 2 shows the reanalysis of FACS-sorted stem/progenitor cells. Purity of the haematopoietic progenitor populations was analyzed on a FACS ARIA II or MFLO XDP after sort. Cells were sorted to at least 99% purity.

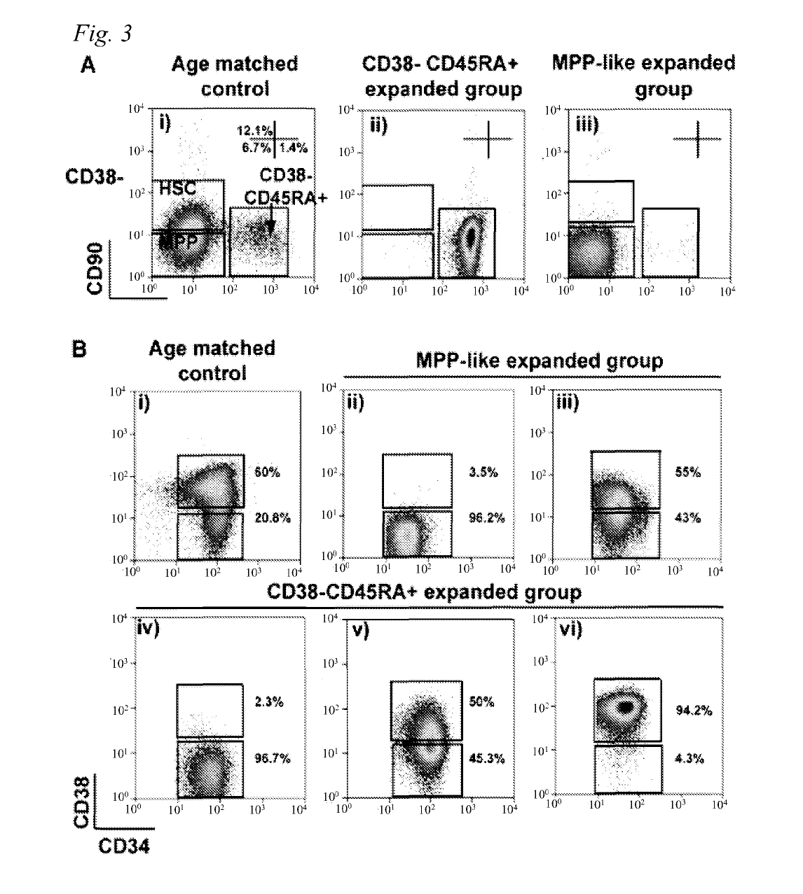

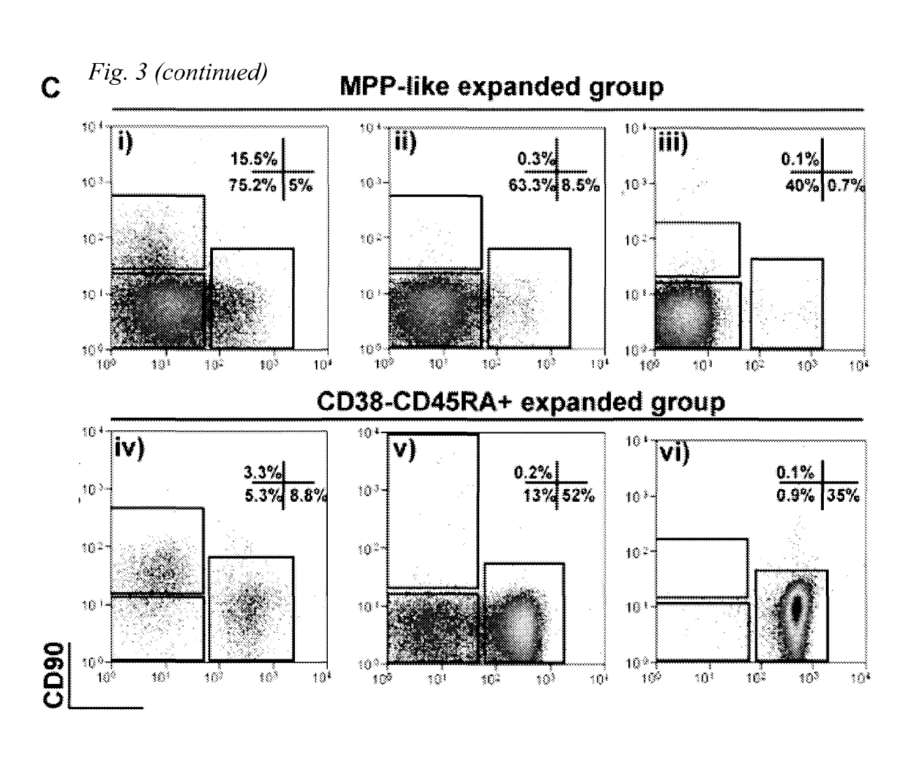

FIG. 3 shows the immunophenotype of stem/progenitor populations in human CD34+ AML. Representative immunophenotypes of CD34+ populations in AML and normal control samples.

(A) CD90 and CD45RA expression in CD34+CD38− cells (top) from control sample (left), an AML sample where the CD45RA+ population is expanded in CD34+CD38− and CD34+CD38+-compartments (centre—45RA+-like expanded) and an AML sample where the CD45RA− population is expanded in the CD34+CD38− and CD34+CD38+ compartments (right—MPP-like expanded).

(B) Different patterns of CD38 expression in CD34+ cells in the MPP-like expanded AML group (top) and 45RA+-like expanded AML group (bottom) with a normal control shown on the top left. The % values are the average for all samples within the group.

(C) Different patterns of CD90 and CD45RA expression in CD34+CD38− cells in MPP-like expanded AML group (top) and 45RA+-like expanded AML group (bottom).

In A-C, the % values are the average for all samples within the group.

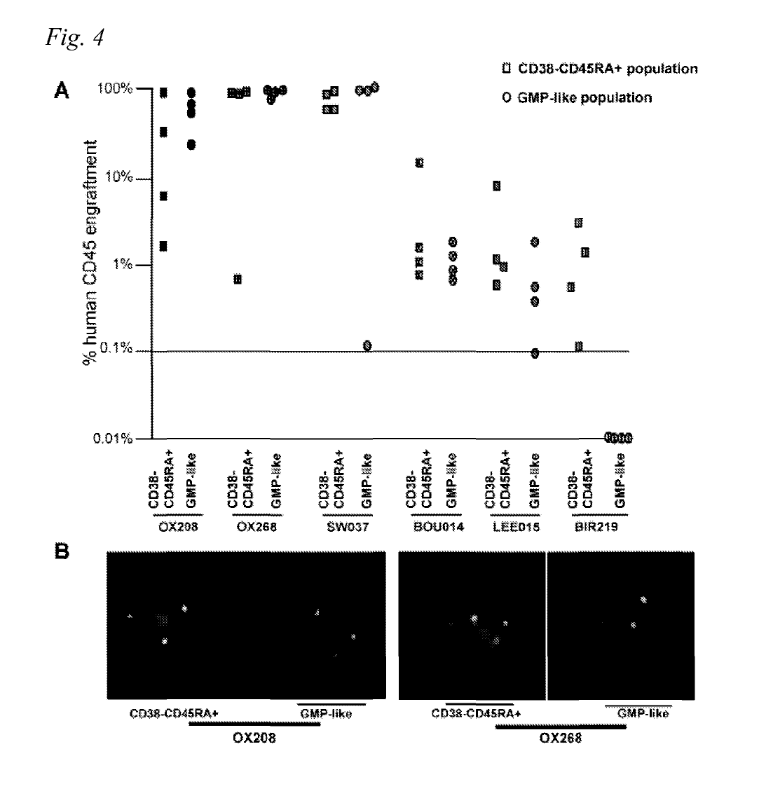

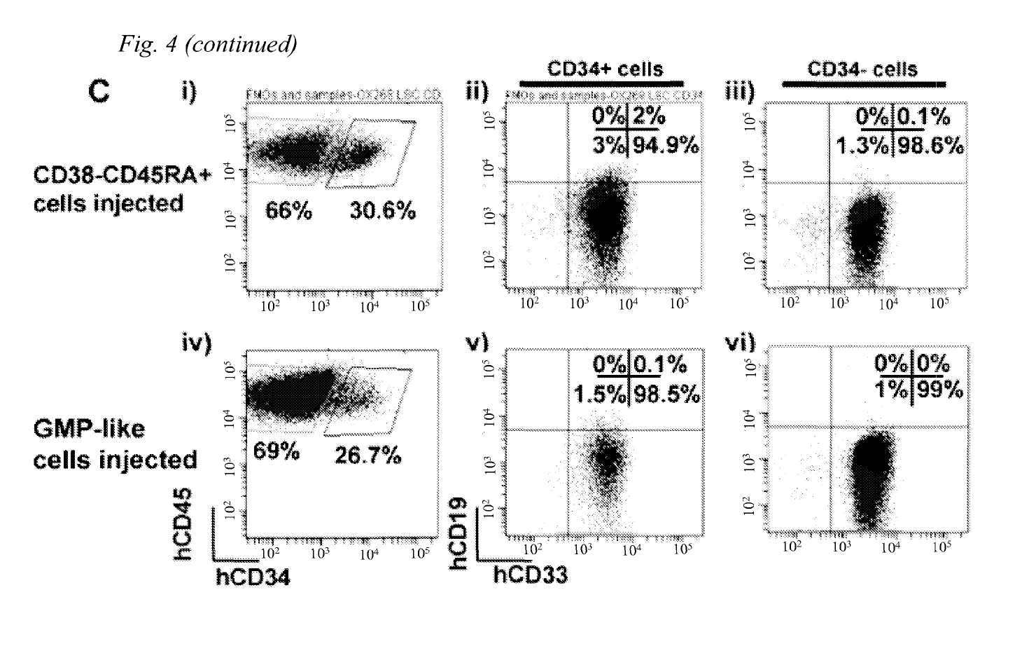

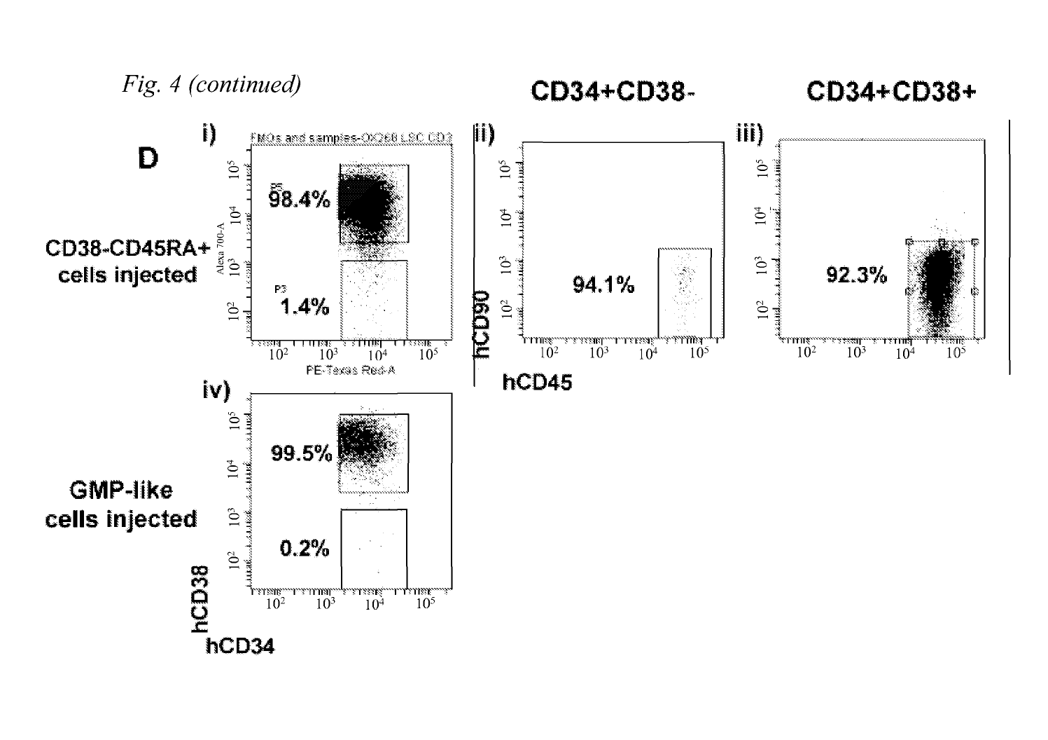

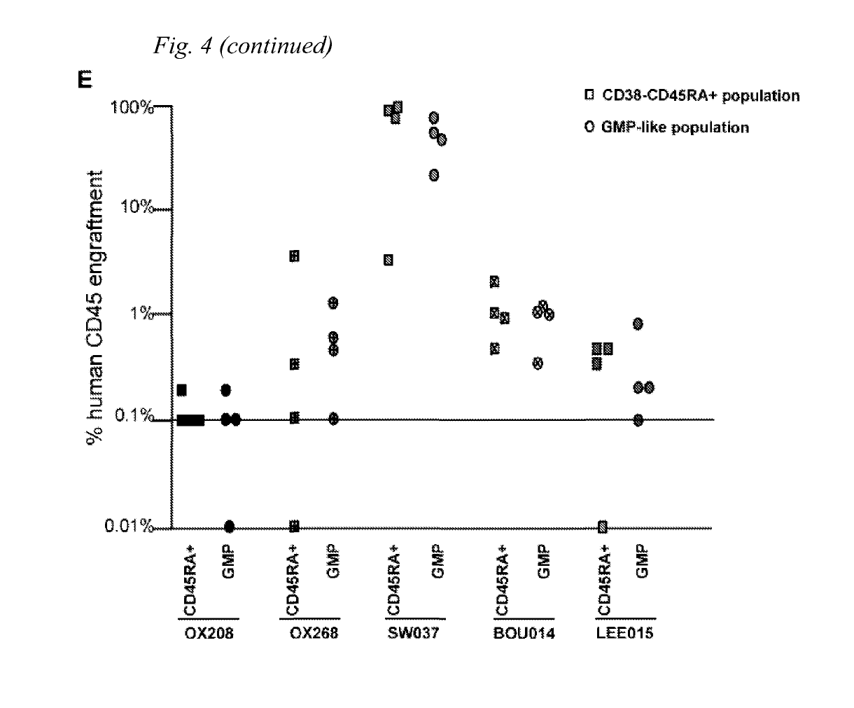

FIG. 4 shows LSC activity in CD45RA+-like expanded AML

(A) % marrow blood engraftment of hCD45RA+ cells of individual NOD-SCID mice 12 weeks after transplantation with 6 different AML patient samples. CD38−CD45RA+ cells (squares) and GMP-like populations (ovals) from each patient were injected into 4 mice.

(B) FISH analysis of engrafted populations from patient OX208 (left) and OX268 (right). In both cases, the cell on the left is from a mouse engrafted with CD38−CD45RA+ cells and the cell on the right engrafted with GMP-like AML population. In patient OX 208, chromosome 14 is detected by a dual colour IGH locus probe and in patient OX268, chromosomes 8 (red signal) and 12 (green) are detected by centromere probes. Cell nuclei are visualized by DAPI counterstain.

(C) A representative example of hCD45RA and hCD34 expression (left, (i) and (v)) and hCD19 and hCD33 expression in hCD34+(centre, (ii) and (v)) and CD34− (right, (iii) and (vi)) populations from primary engrafted mice when transplanted with CD38−CD45RA+ (top) and GMP-like (bottom) AML populations. The mean % values from all 6 patients are shown.

(D) A representative example of hCD38 and hCD34 expression on engrafted hCD34+ cells (left, (i) and (iv)), hCD90 and hCD45RA expression in hCD34+CD38− cells (centre, (ii)) when CD38−CD45RA+ cells (top) and GMP-like AML (bottom) cells are injected into primary engrafted mice. The mean % values from all 6 patients are shown.

(E) % engraftment of hCD45RA+ cells in marrow of individual in secondary transplanted mice 12 weeks after intravenous injection with pooled hCD45+ cells from primary engrafted mice initially injected with either CD38−CD45RA+ (squares) and GMP-like (oval) cells. 4 secondary recipient mice were injected per population. Pooled cells from each primary leukaemia are shown as a different symbol.

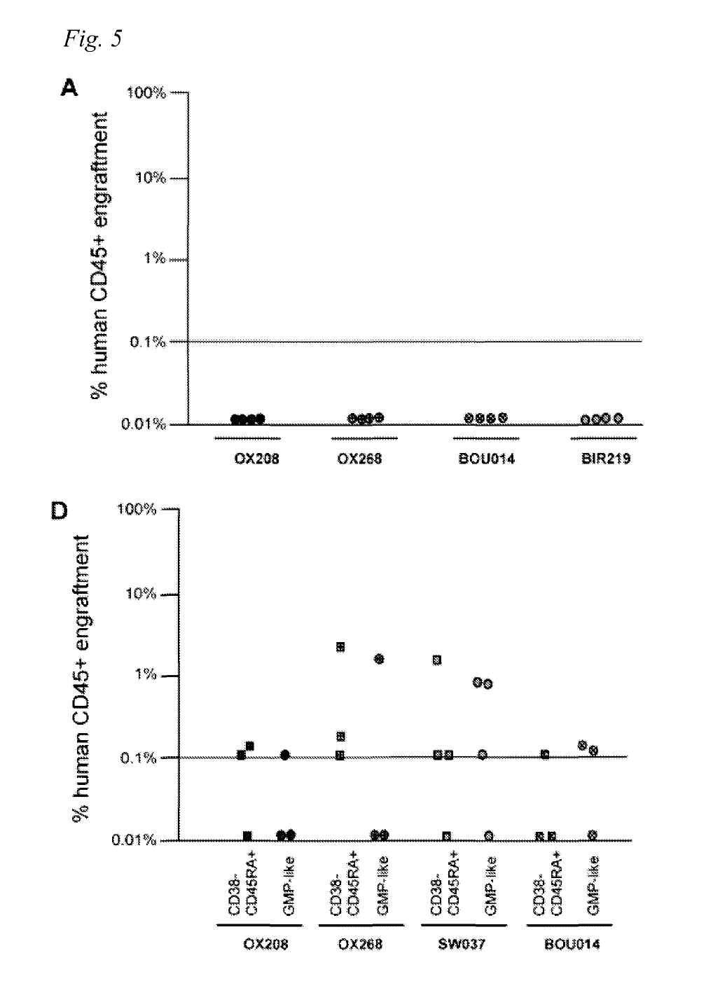

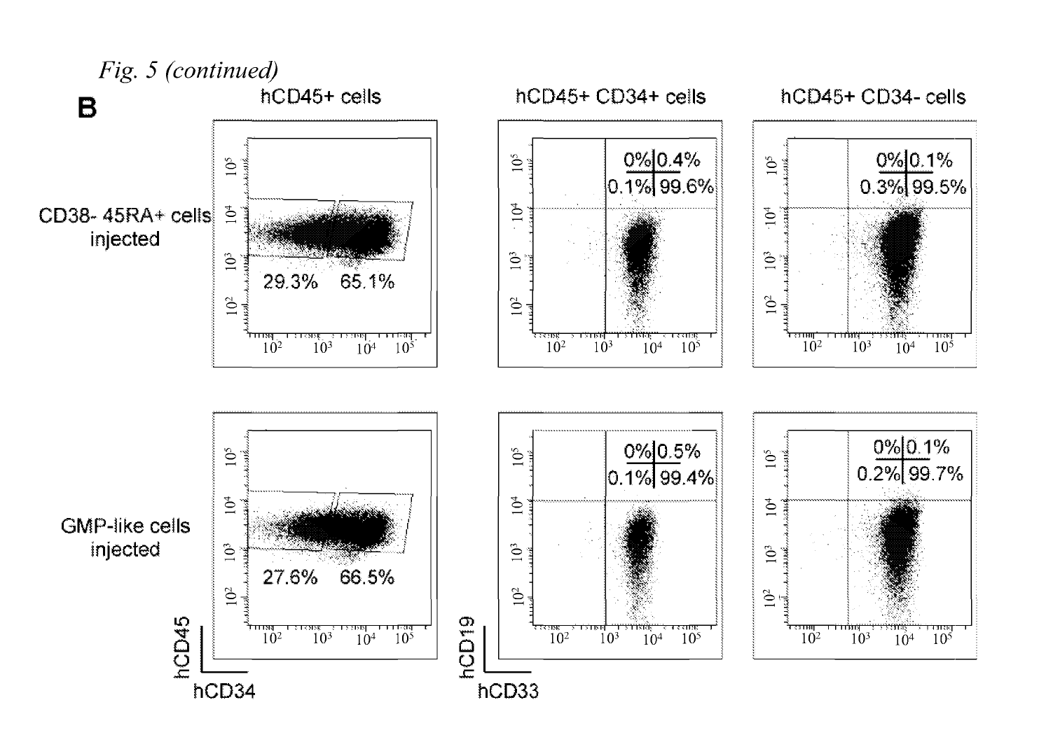

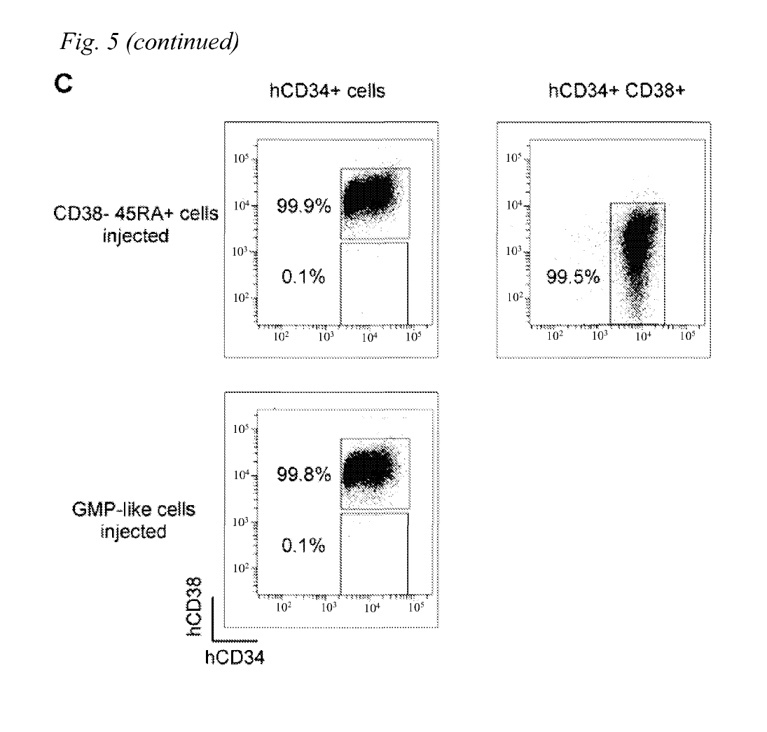

FIG. 5 shows immunophenotypic analysis of engrafted human cells in secondary xenotransplanted animals.

(A) % marrow blood engraftment of hCD45RA+ cells of individual NOD-SCID mice 12 weeks after transplantation with 4 different AML patient samples.

(B) A representative example of human (h) CD38 and CD34 expression of engrafted hCD34+ cells (left), hCD90 and hCD45RA expression in hCD34+CD38− cells (centre) and hCD110 and hCD45RA expression in hCD34+CD38+ cells when total CD45+ cells are injected into secondary recipient mice after primary engraftment of CD34+ CD38− CD45RA+(top) or CD34+ CD38+ CD110− CD45RA+ (bottom) cells. The % values are the mean from all patients.

(C) A representative example of hCD45RA and hCD34 expression (left) and hCD19 and hCD33 expression in hCD34+(centre) and CD34− (right) populations when total CD45+ cells are injected into secondary recipient mice after primary engraftment of CD34+ CD38− CD45RA+ (top) or CD34+ CD38+ CD110− CD45RA+ (bottom) cells. The % values are the mean from all patients.

(D) % marrow blood engraftment of hCD45RA+ cells of individual NOD-SCID mice 12 weeks after transplantation with 4 different AML patient samples.

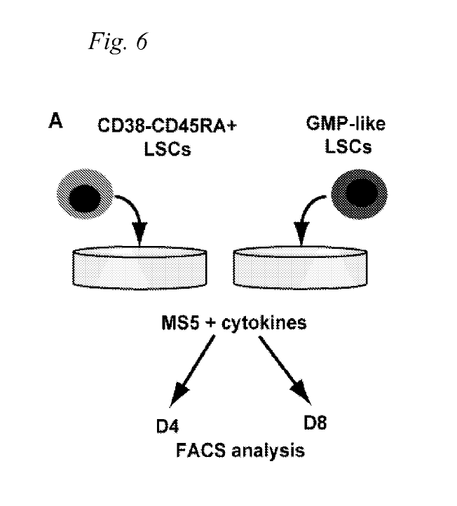

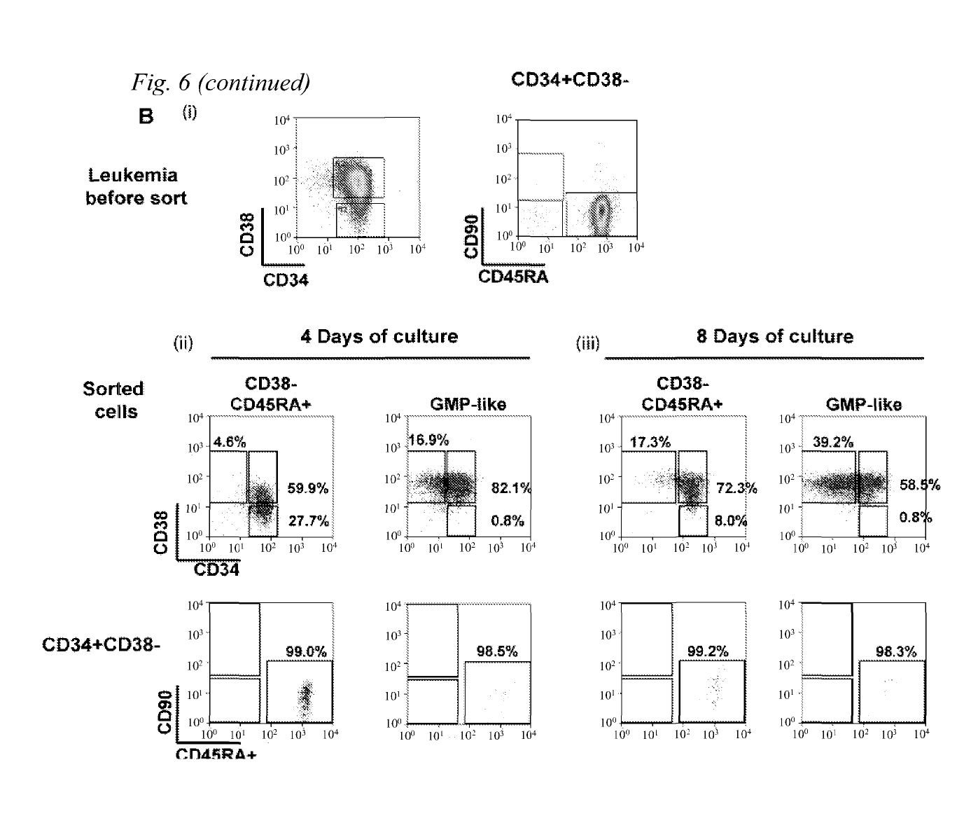

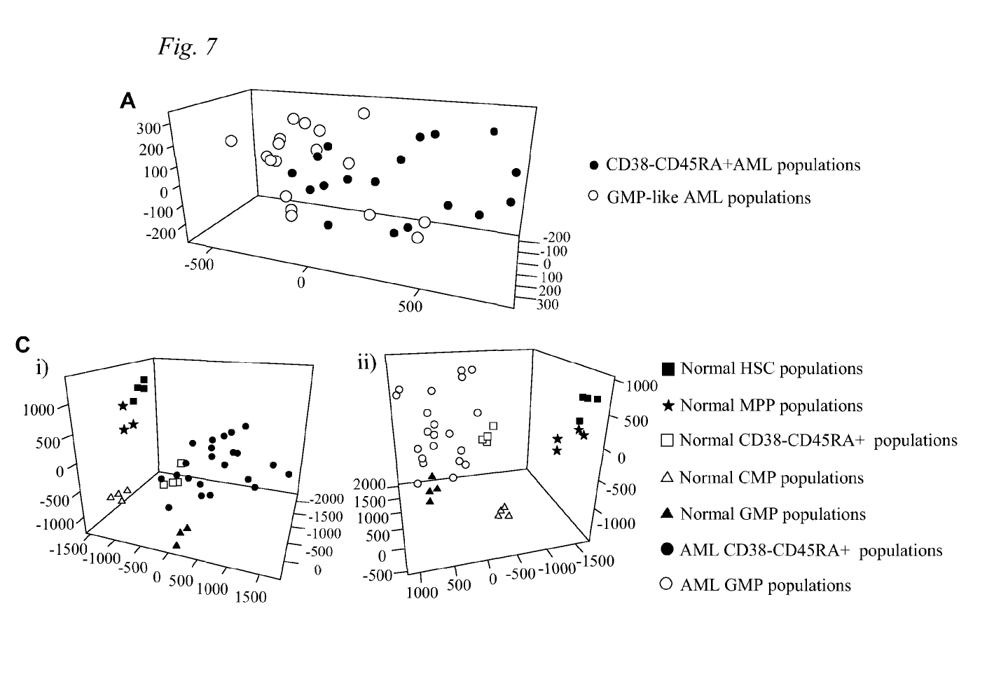

FIG. 6 shows in vitro hierarchy in CD45RA+ like expanded AML.

(A) Schematic experimental outline. FACS-sorted CD38−CD45RA+ or GMP-like populations from 5 AML samples were separately cultured for either 4, or 8 days, and then analyzed by FACS.

(B) (i) Top left, representative example of CD34 and CD38 expression in an AML sample. The purity of CD38−CD45RA+ (right) populations post-FACS sort and prior to culture is shown.

Below, representative FACS analysis of cell populations when CD38−CD45RA+ and GMP-like populations have been cultured for either 4 days (ii—left) or 8 days (iii—right). Top panel, CD34 and CD38 expression; bottom panel, CD90 and CD45RA expression in CD34+CD38− cells. The percentages shown are the mean for gated populations for all 5 AML samples studied.

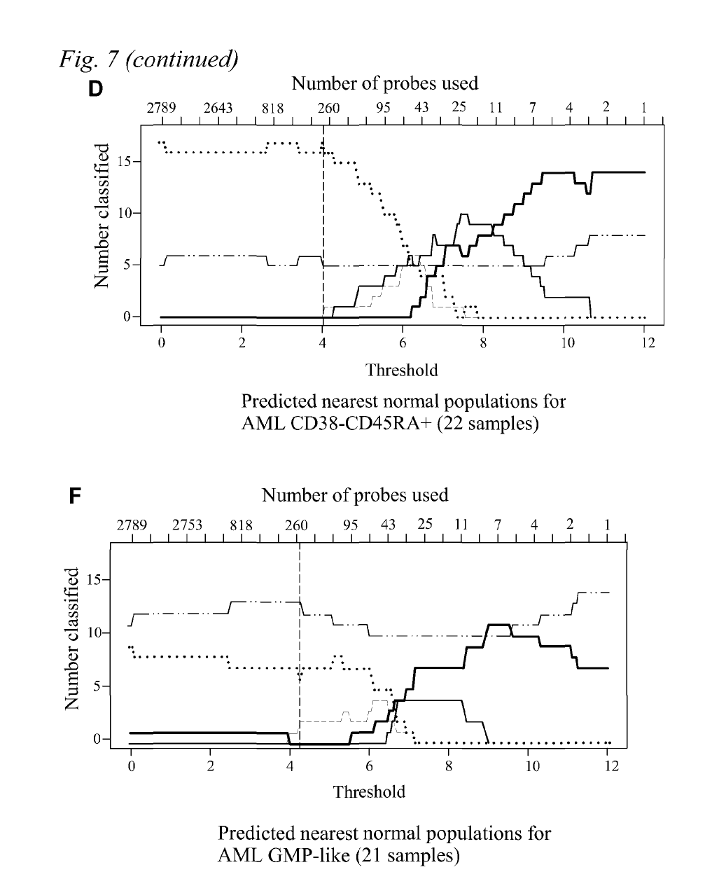

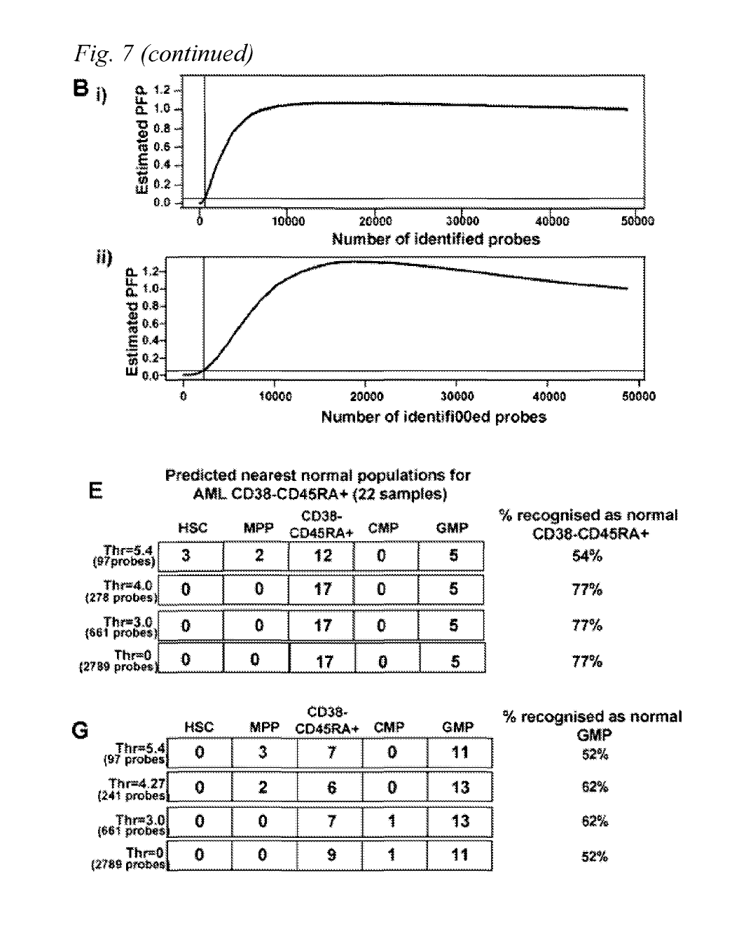

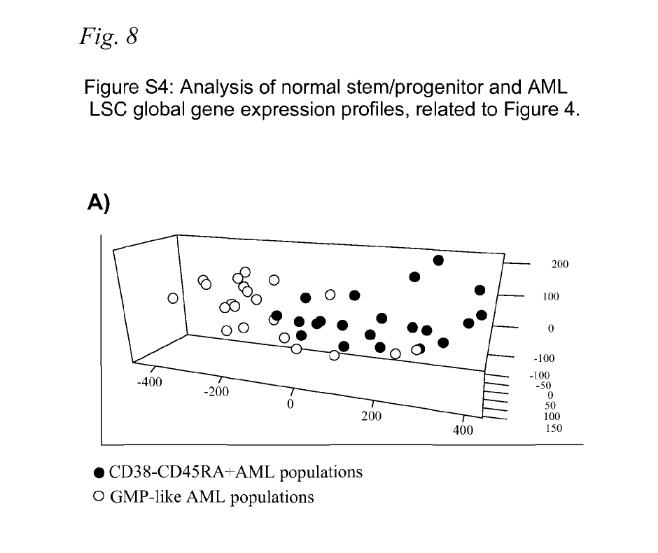

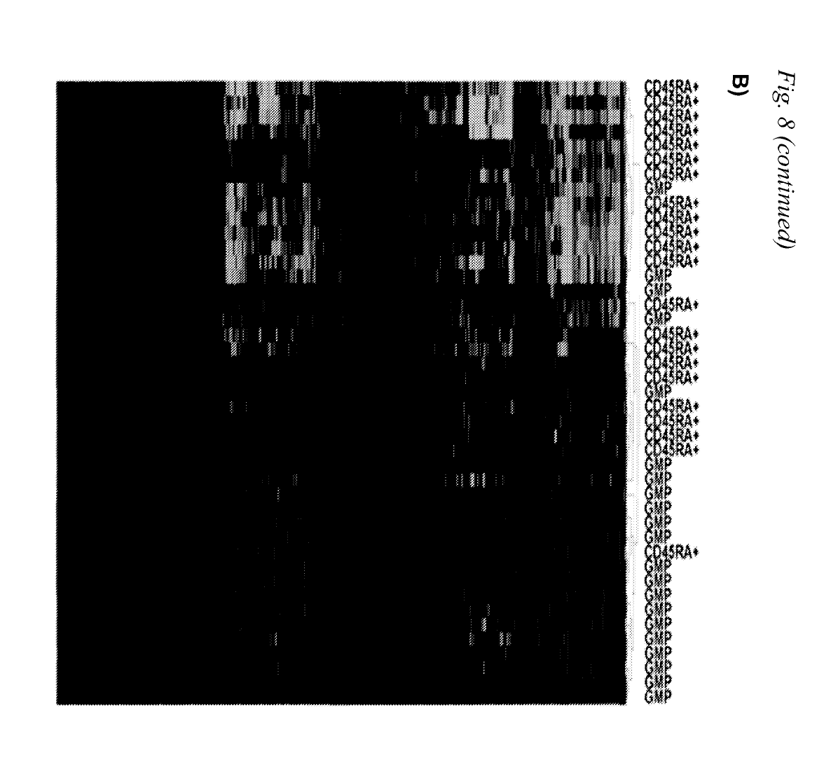

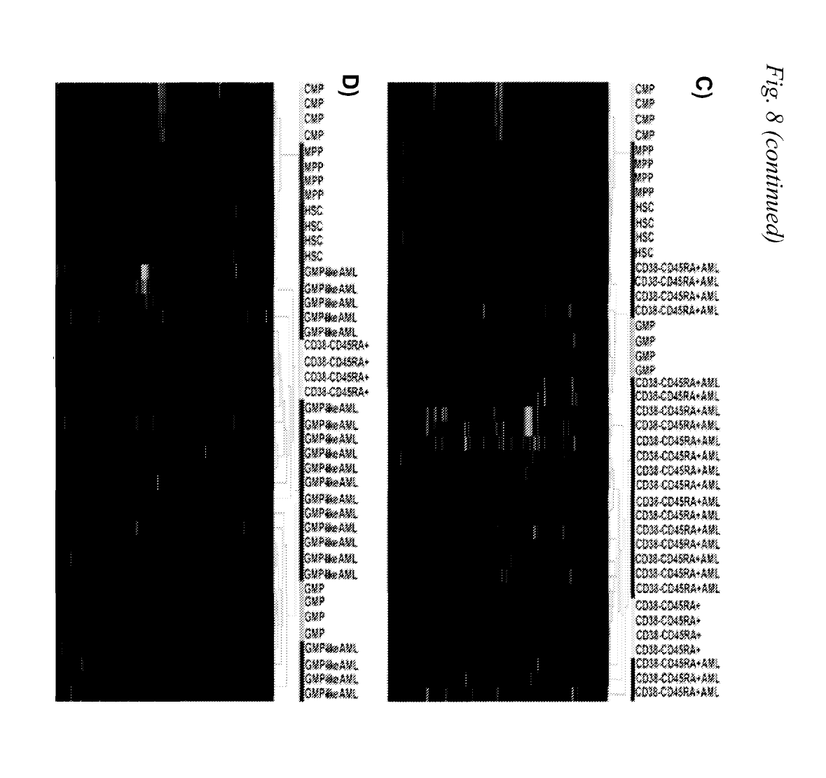

FIG. 7 shows gene expression of AML LSC populations and normal stem and progenitor populations.

(A) 3-D Principle Component Analysis (PCA) displaying gene expression profiles using 917 differentially expressed probes (corresponding to 748 mapped genes) determined by a paired t-test with a cut-off of 0.01 from 18 AML patients where both CD38−CD45RA+ (blue spheres) and GMP-like populations (red spheres) were available from the same patient.

(B) Rank product analysis of probe sets showing either increased expression in CD38−CD45RA+ compared to GMP-like populations (i) and vice versa (ii) using gene expression profiles from 18 AML patients where both CD38−CD45RA+ and GMP-like population samples were available from the same patient. On the y-axis, the false discovery rate; on the x-axis the rank product of the probes. At a false discovery rate of 0.05, the numbers of differentially expressed probes are shown as red lines.

(C) 3-D Principal Component Analysis of expression profiles of FACS-sorted 4 normal populations (HSC—black spheres, MPP—brown spheres, CD34+CD38− CD90−CD45RA+—yellow spheres, CMP—pink spheres and GMP—green spheres) using a 2629 ANOVA gene set (2789 probes) of differentially expressed genes in the normal populations. The positions of 22 CD38−CD45RA+ AML populations (blue spheres) (left) and 21 GMP-like AML populations (red spheres) (right) are shown.

(D-G) Classifier analysis using the 2789 ANOVA selected differentially expressed set of probes that separates normal populations. This probe set was used to call the identity of the 22 CD38−CD45RA+ AML populations (D-E) and 21 GMP-like AML populations (F-G). On the x-axis more probes (from the most differentially expressed to the least differentially expressed) are used in the classifier from right to left. The top x-axis shows the numbers of probes used. The bottom x-axis depicts the corresponding threshold values. Y-axis, the number of AML samples called.

FIG. 8 shows the analysis of stem/progenitor expression profiles in AML LSC and normal stem/progenitor cells.

(A) Non-paired t-test was used to identify a gene set differentially expressed between 22 CD38−CD45RA+ and 21 GMP-like AML populations. This gene set was used to display gene expression in a 3-D PCA analysis. Gene expression in each CD38−CD45RA+ AML sample is shown blue sphere and that in each GMP-like AML sample as a red sphere.

(B) Hierarchical clustering analysis of gene expression of 22 CD38−CD45RA+ and 21 GMP-like AMP populations using the set of differentially expressed genes identified by t-test analysis.

(C and D) Hierarchical clustering analysis to show the relationship between normal HSC/term populations and CD38−CD45RA+ (C) and GMP-like AML populations using an Anova gene set of 2789 genes that maximally differentiates normal HSC/progenitor populations by gene expression.

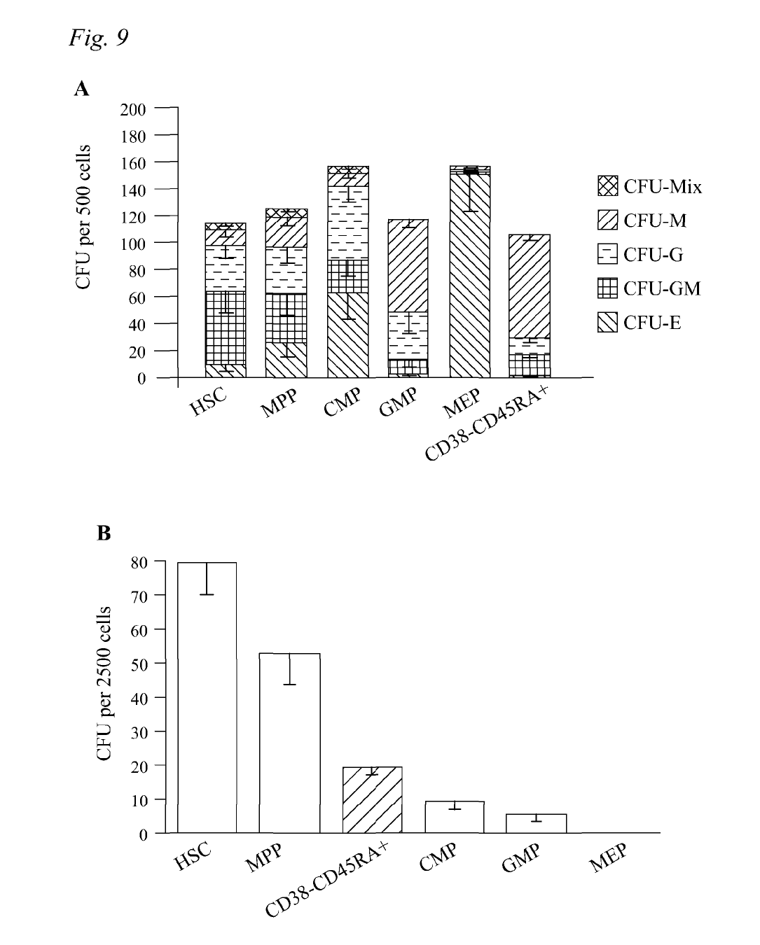

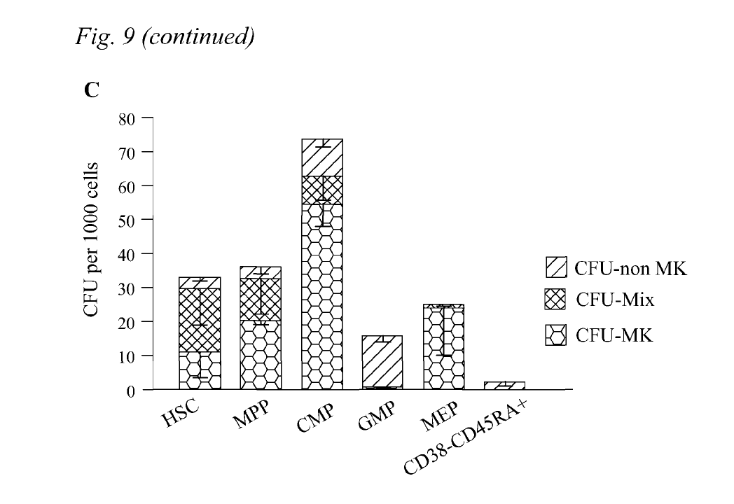

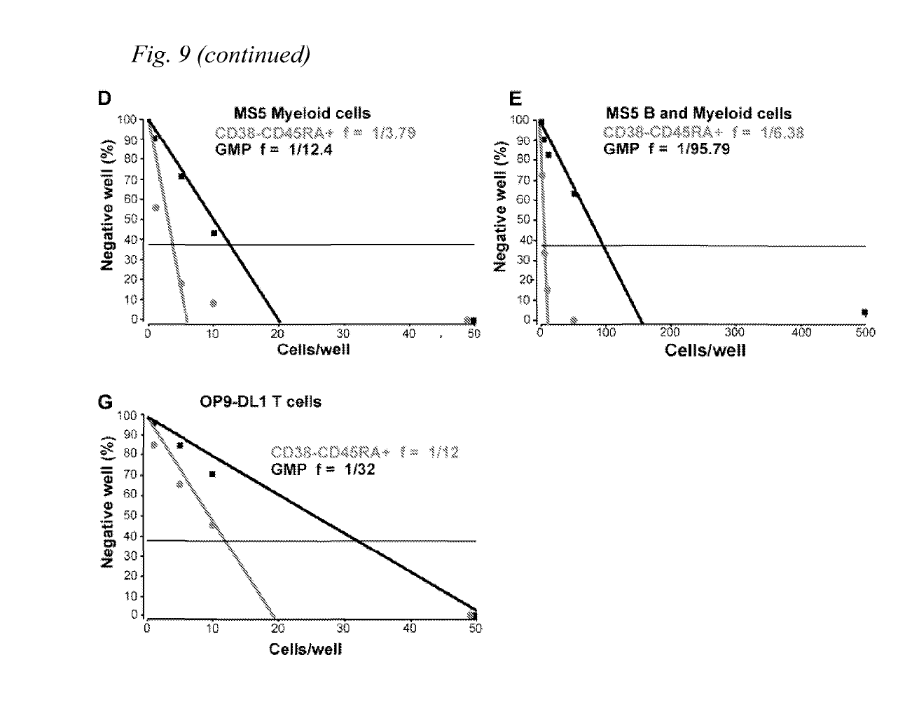

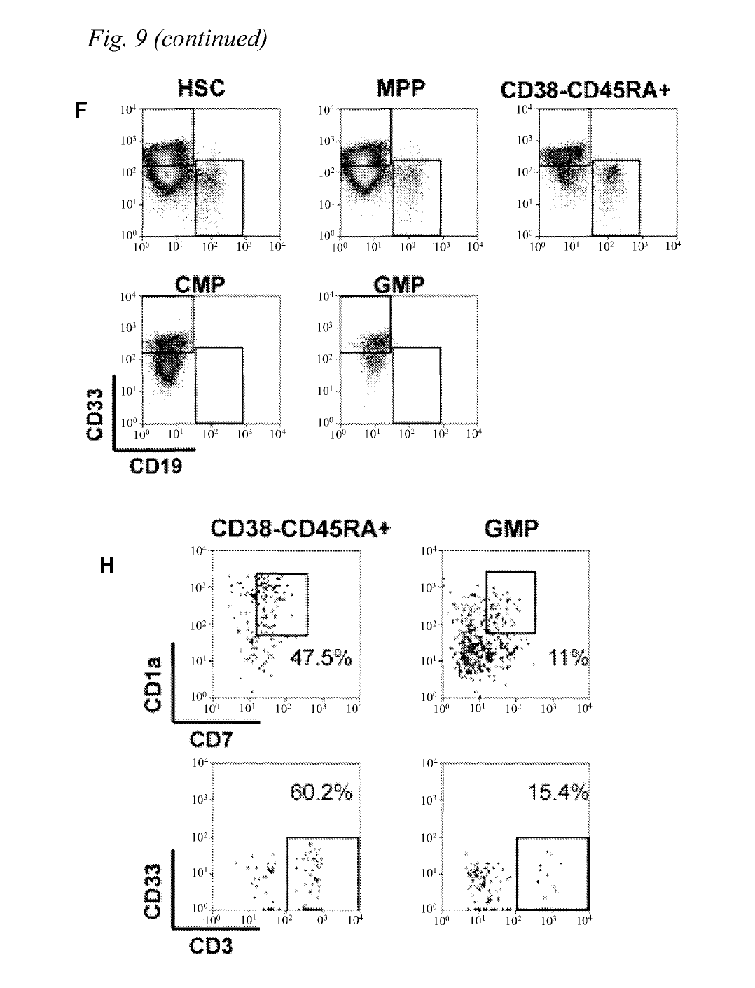

FIG. 9 shows that normal CD38−CD45RA+ cells have lymphoid primed multipotential myeloid potential.

(A) Myeloid/erythroid colony growth of FACS sorted normal marrow HSC, MPP, CMP, GMP, MEP and CD38−CD45RA+ populations from 5 normal samples. Number of colonies/500 cells plated were scored (mean±1 S.D.). Colony lineage affiliation is shown by different colored bars (right of panel).

(B) Cells from colonies in the primary platings (A) were plated in a secondary replating assay. The number of colonies/2500 cells plated from each of the cell type is illustrated (mean±1 S.D.).

(C) Megakaryocyte colony growth from FACS sorted normal marrow HSC, MPP, CMP, GMP, MEP and CD38−CD45RA+ populations from 3 normal individuals. The number of colonies/1000 cells plated is depicted. Colony lineage affiliation is shown by different colored bars (right of panel).

(D and E) Limit dilution analysis to determine frequency of cells with myeloid potential (D) and mixed B-cell/myeloid potential (E) in CD38−CD45RA+ (CD45RA+) (green line) and GMP cells (blue line). 1-500 sorted cells from 4 normal marrow samples were tested in individual wells in MS5 stroma/cytokine co-culture (144 replicates for each condition). Cell output was analyzed by FACS.

(F) Representative FACS analysis of CD19 and CD33 expression in cells produced from bulk culture of FACS sorted HSC, MPP, CD38−CD45RA+, CMP and GMP cells from 4 normal marrows.

(G) Limit dilution analysis to determine T-cell potential frequency in CD38− CD45RA+ (green line) and GMP cells (blue line). 1-500 sorted cells from 3 marrow samples were tested in individual wells in OP-DL1 stroma/cytokine co-culture (36 replicates for each condition). Cell output was analyzed by FACS.

(H) Representative FACS analysis plots of CD1a, CD7 (top) and CD33 and CD3 expression in cells produced from bulk culture of FACS sorted CD38−CD45RA+ and GMP cells from 3 marrows.

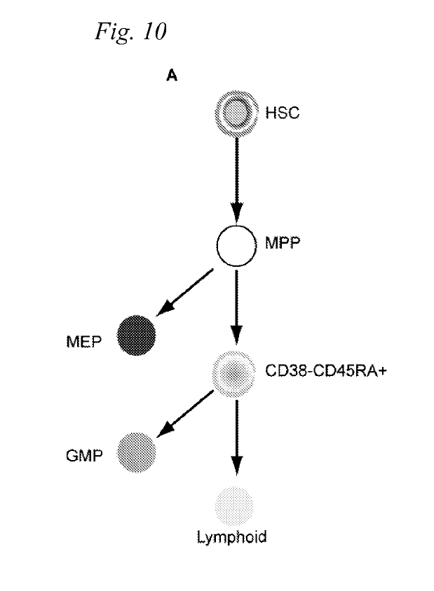

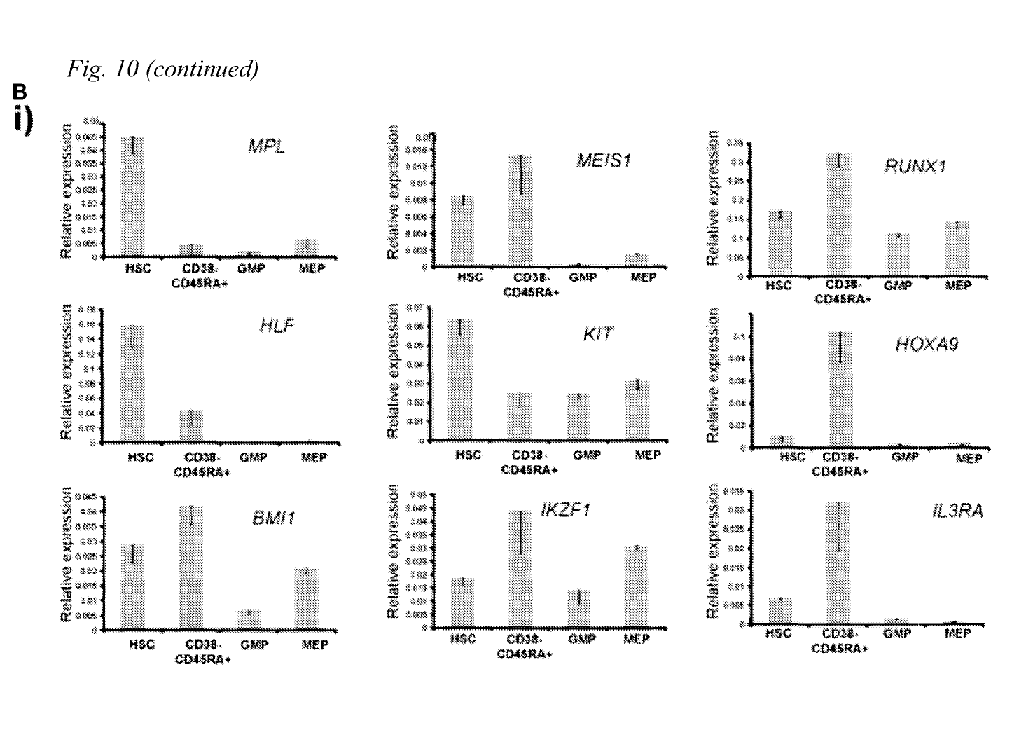

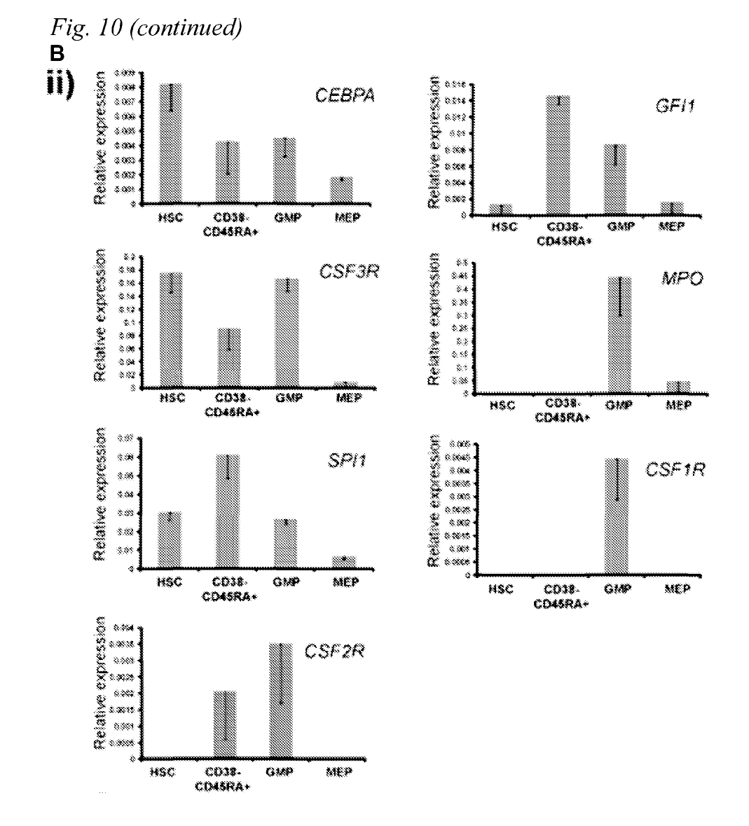

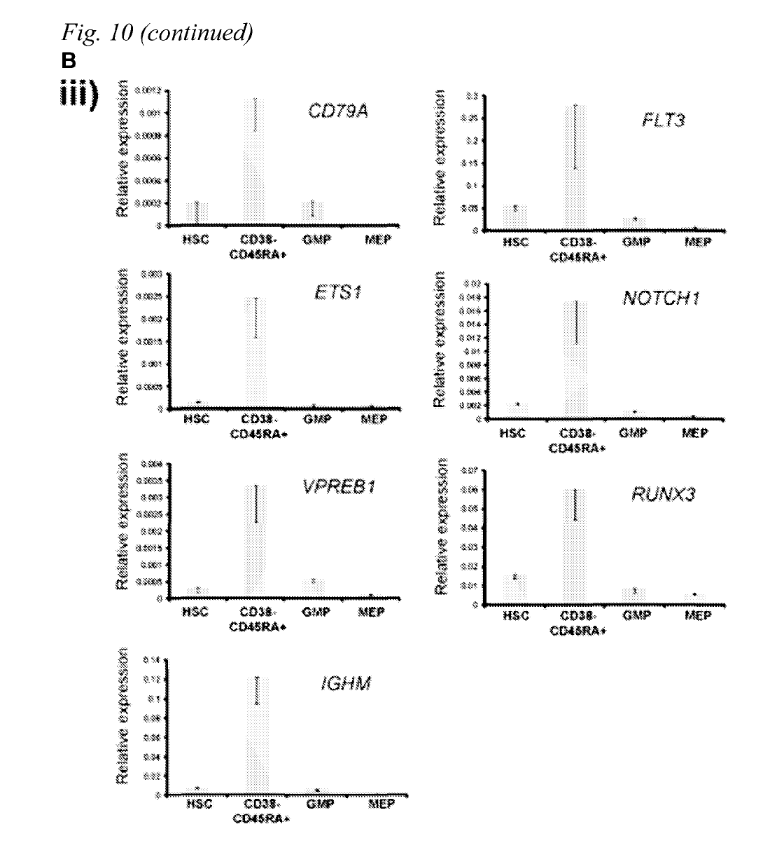

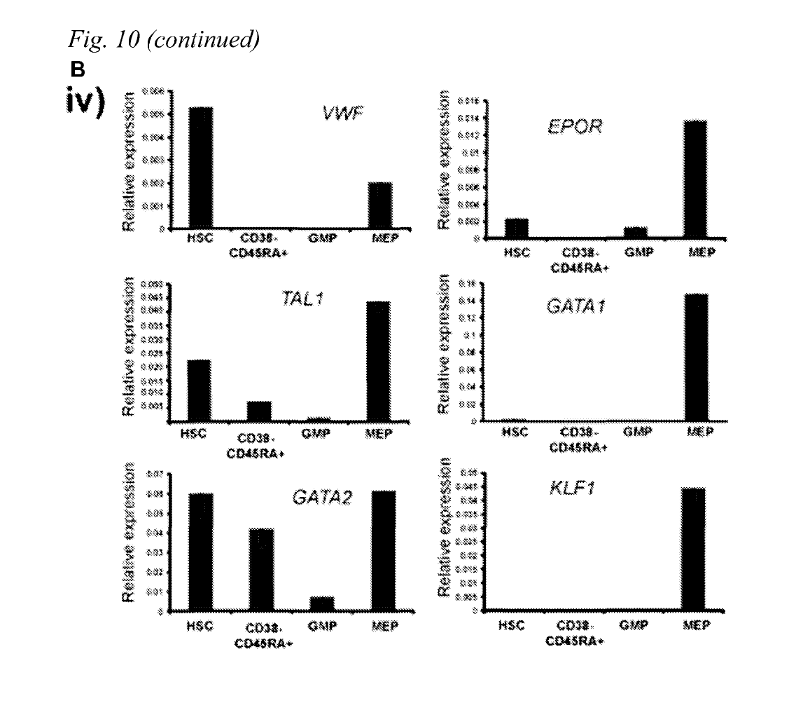

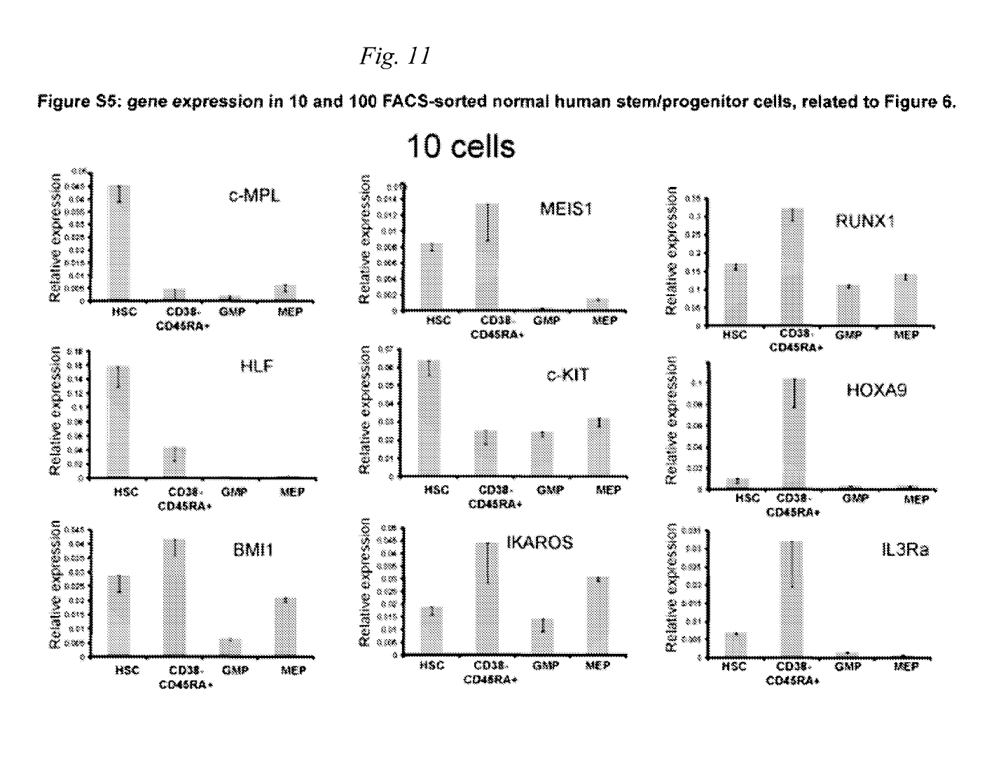

FIG. 10 demonstrates granulocyte-monocyte and lymphoid but not erythroid-megakaryocyte gene expression in CD34+CD38−CD90−CD45RA+ cells.

(A) Schematic diagram of the cellular hierarchy of human stem/progenitor cells and the cascade of lineage-affiliated gene signatures. HSC, haematopoietic stem cells; MPP, multi-potential cells; CD38−CD45RA+, Lin-CD34+CD38−CD90− CD45RA+ cells; MEP, megakaryocytic-erythroid progenitor; GMP, granulocyte-macrophage progenitor. HSC- and CD38−CD45RA+-affiliated gene signature (green); myeloid (GM) lineage-affiliated gene signature (blue); lymphoid lineage-affiliated gene signature (yellow); ME lineage-affiliated gene signature (red).

(B) Mean mRNA expression (±1 SD) of indicated genes relative to GAPDH was determined in 5 replicates by Quantitative Real-Time RT-PCR in 10 FACS-sorted HSC, CD38−CD45RA+, GMP and MEP cells from 2 independent control normal samples. Data from genes affiliated with: (i) HSC and CD38−CD45RA+ cells (green bars); (ii) granulocyte-monocyte lineage cells (blue bars); (iii) lymphoid lineage cells (yellow bars); (iv) erythroid-megakaryocyte lineage cells (red bars).

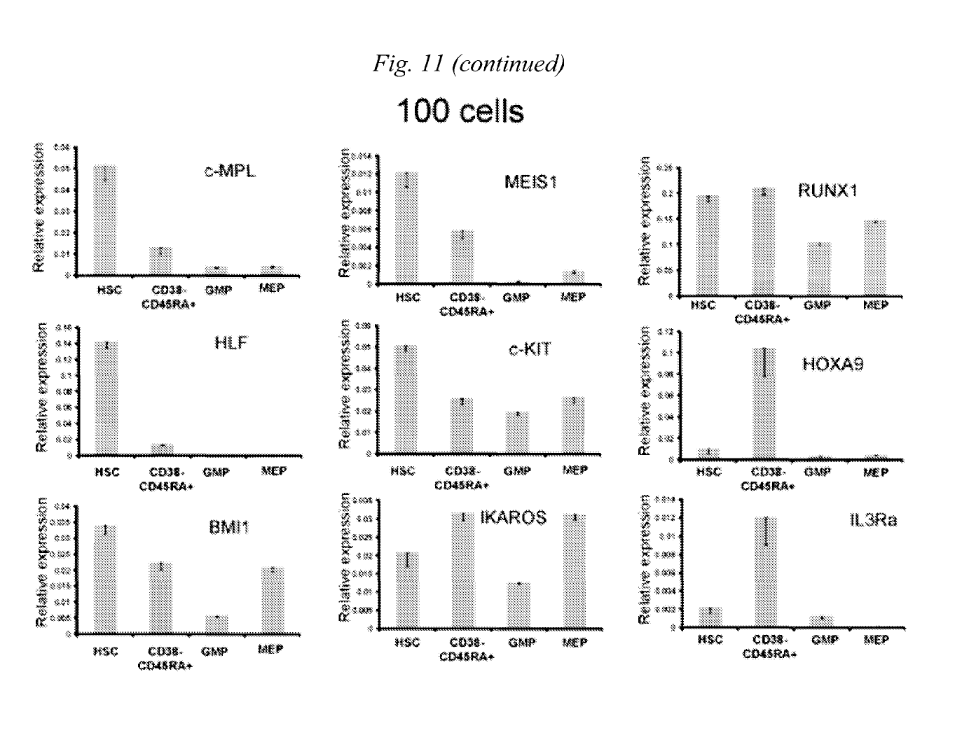

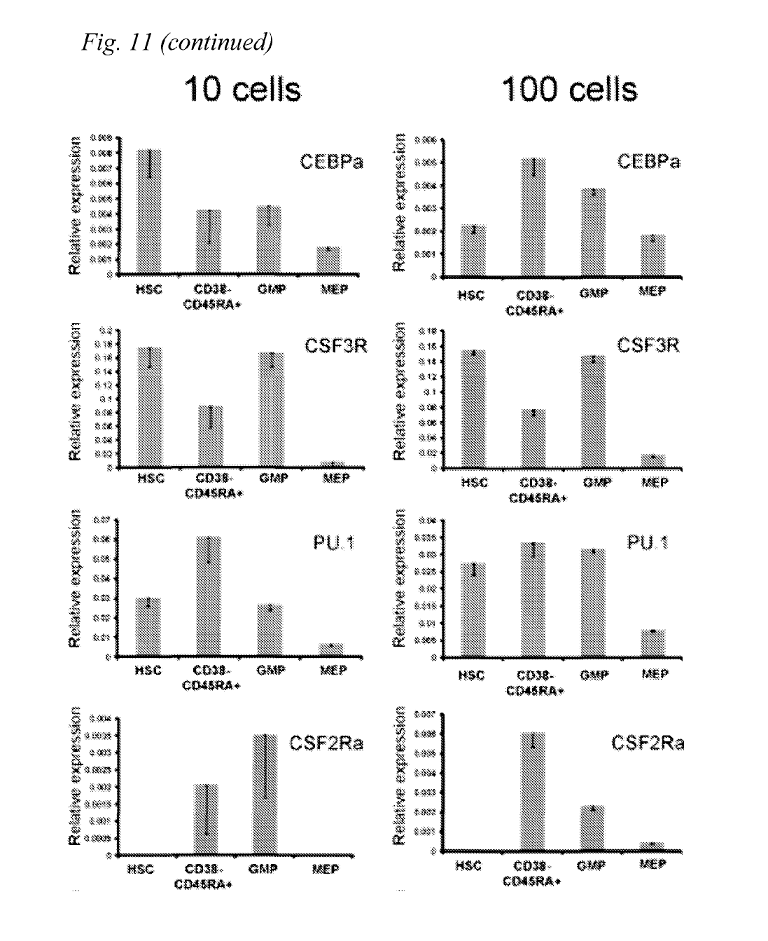

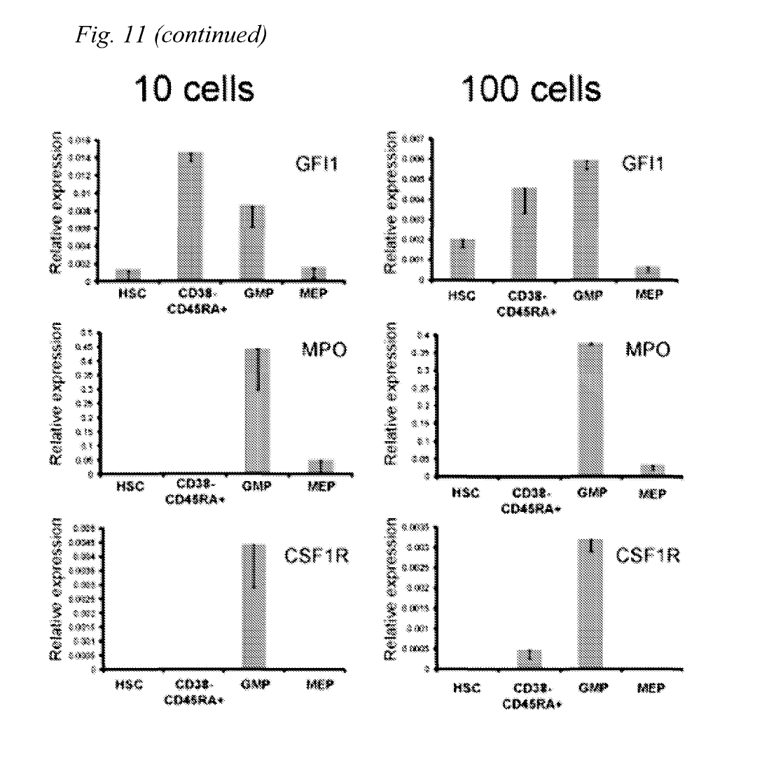

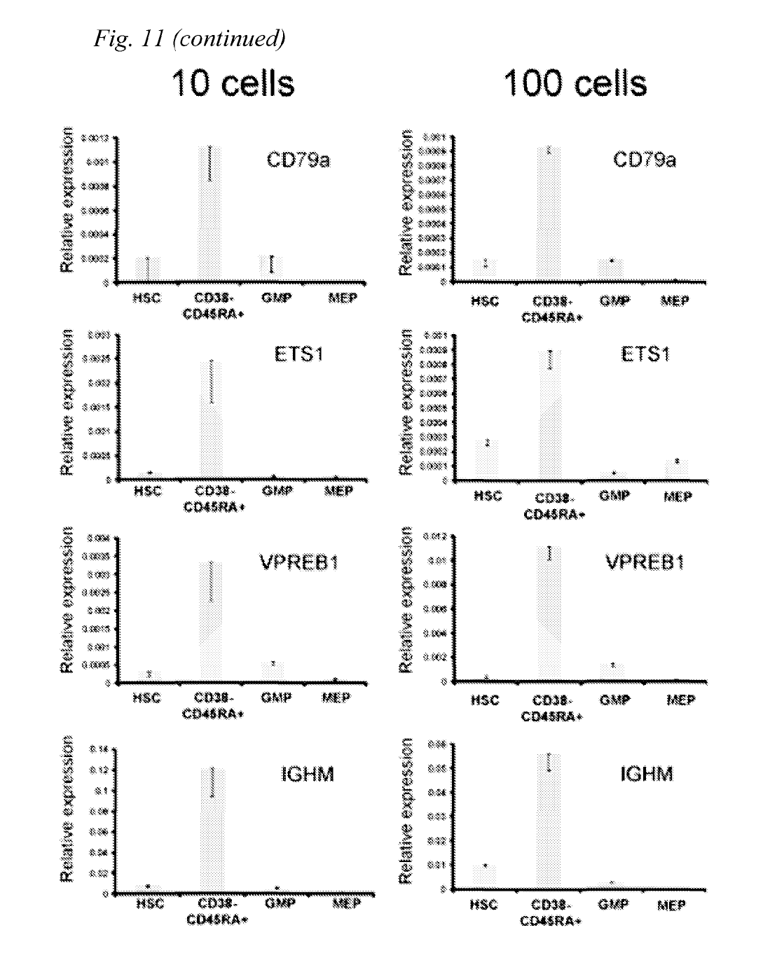

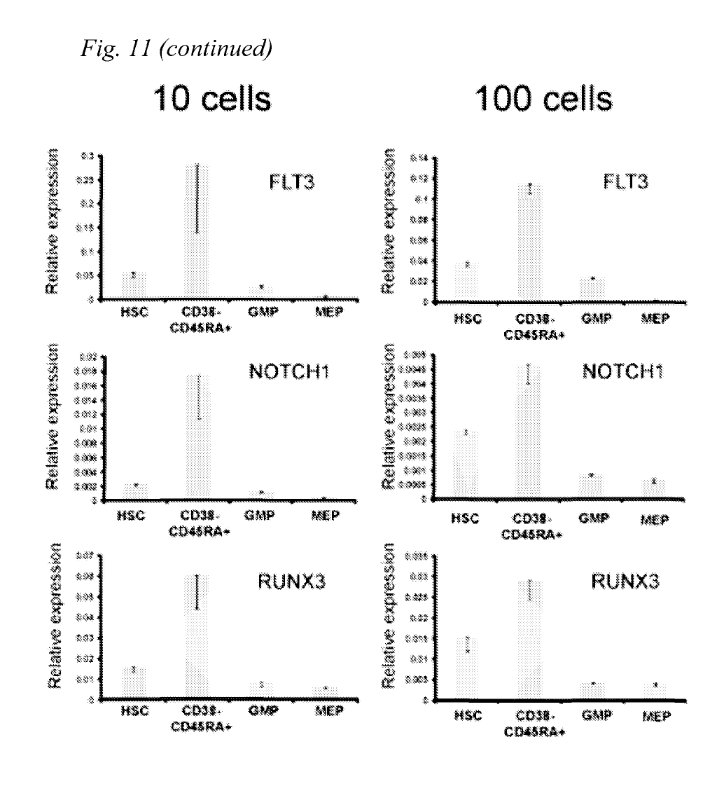

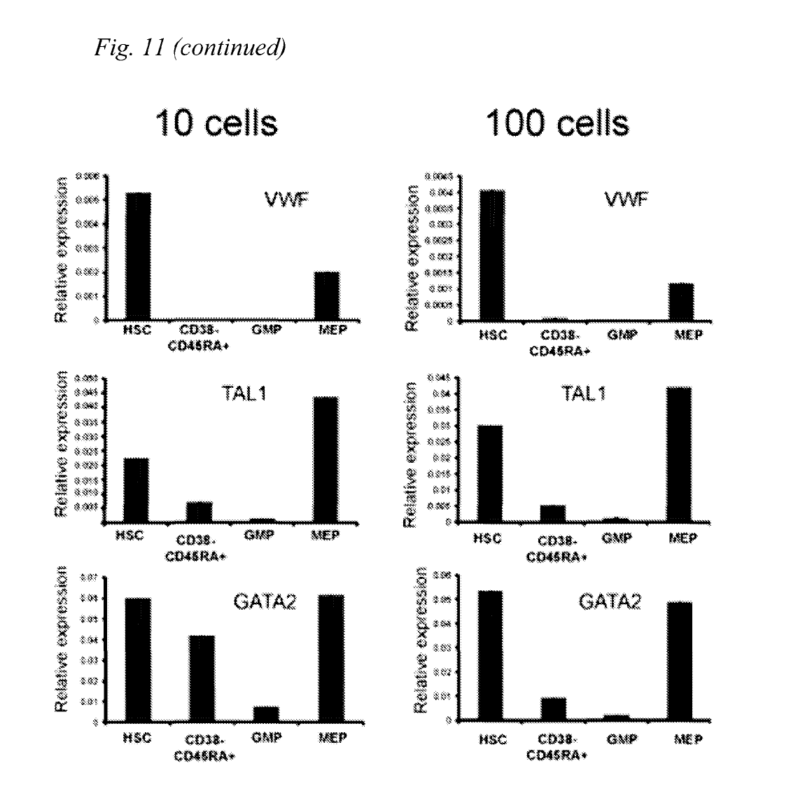

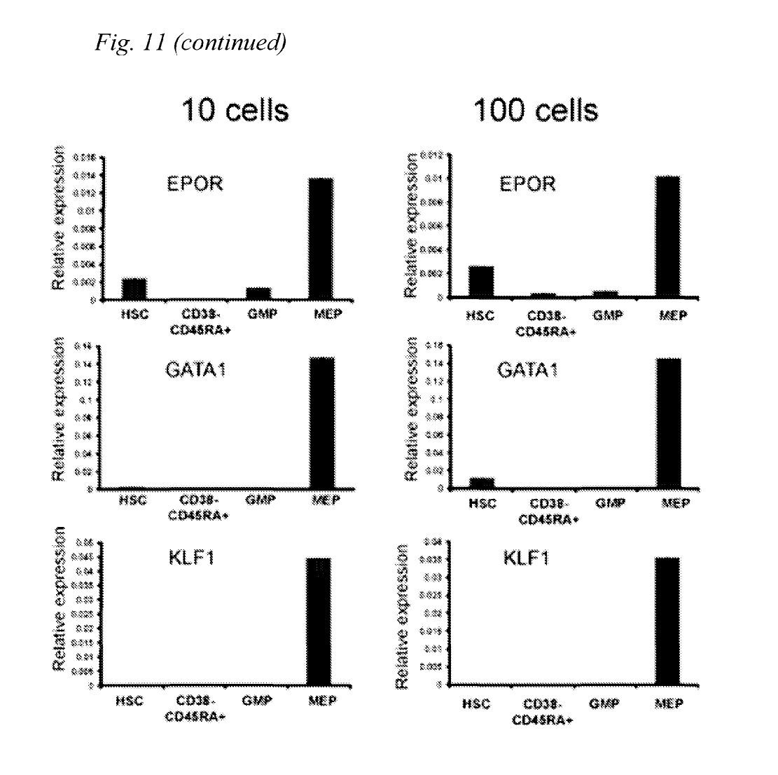

FIG. 11 shows gene expression data in highly purified normal stem/progenitor cells.

Multiplex quantitative PCR data of indicated genes on FACS sorted HSC, CD38− CD45RA+, GMP and MEP cells. All data were normalized to the expression of GAPDH. Results represent the mean value from 5 replicates from 2 control samples. Data from genes affiliated with: (i) HSC and CD38−CD45RA+ cells (green bars); (ii) granulocyte-monocyte lineage cells (blue bars); (iii) lymphoid lineage cells (yellow bars); (iv) erythroid-megakaryocyte lineage cells (red bars).

FIG. 12 shows the results of demographic and immunophenotypic characteristics associated with samples used in xenotransplantation experiments, related to FIG. 4

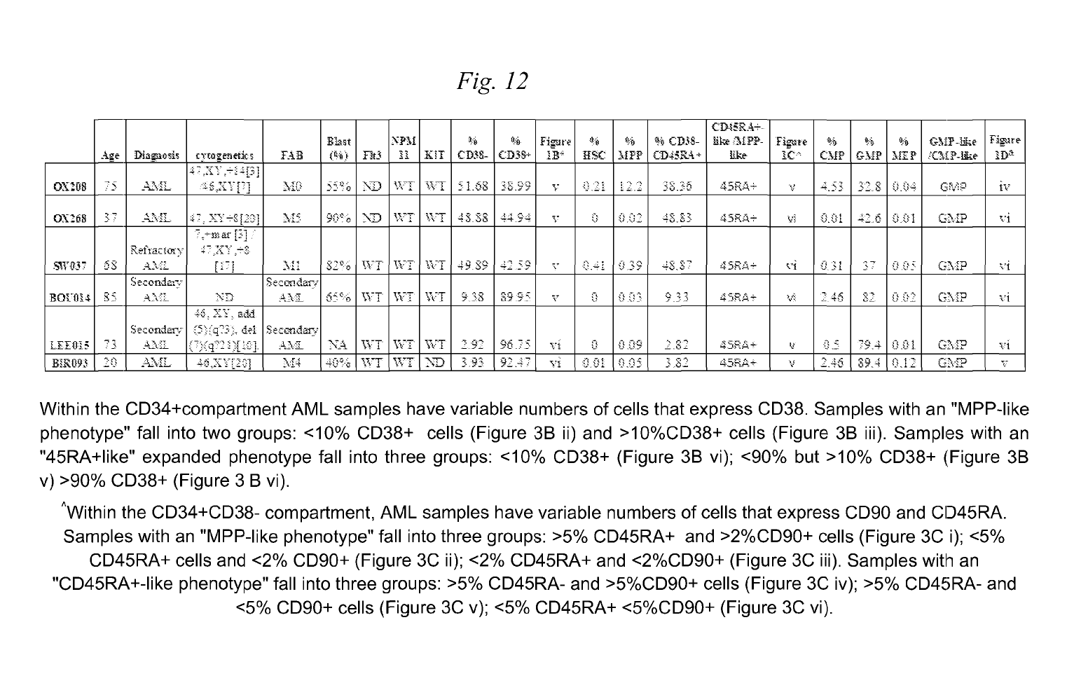

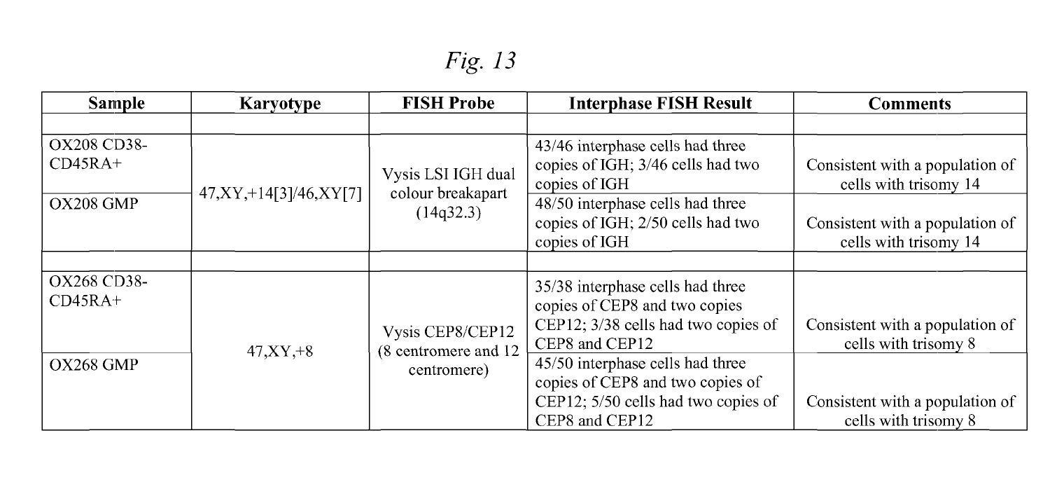

FIG. 13 shows the results of cytogenetic analysis of FACS-sorted engrafted human cells in primary xenotransplant animals, related to FIG. 4.

### KEY TO SEQ ID NOs

SEQ ID NO: 1 CD34 amino acid sequence
SEQ ID NO: 2 CD45RA amino acid sequence
SEQ ID NO: 3 CD90 amino acid sequence
SEQ ID NO: 4 CD123 amino acid sequence
SEQ ID NO: 5 CD38 amino acid sequence
SEQ ID NO: 6 CD19 amino acid sequence
SEQ ID NO: 7 CD47 amino acid sequence
SEQ ID NO: 8 CCR8 amino acid sequence
SEQ ID NO: 9 RHAMM amino acid sequence
SEQ ID NO: 10 CD86 amino acid sequence
SEQ ID NO: 11 CD34 nucleic acid sequence
SEQ ID NO: 12 CD45RA nucleic acid sequence
SEQ ID NO: 13 CD90 nucleic acid sequence
SEQ ID NO: 14 CD123 nucleic acid sequence
SEQ ID NO: 15 CD38 nucleic acid sequence
SEQ ID NO: 16 CD19 nucleic acid sequence
SEQ ID NO: 17 CD47 nucleic acid sequence
SEQ ID NO: 18 CCR8 nucleic acid sequence
SEQ ID NO: 19 RHAMM nucleic acid sequence
SEQ ID NO: 20 CD86 nucleic acid sequence

SEQUENCE LISTING:

SEQ ID NO: 1
mlvrrgarag prmprgwtal cllsllpsgf msldnngtat pelptqgtfs nvstnvsyqe
tttpstlgst slhpvsqhgn eattnitett vkftstsvit svygntnssv qsqtsvistv
fttpanvstp ettlkpslsp gnvsdlstts tslatsptkp ytssspilsd ikaeikcsgi
revkltqgic leqnktssca efkkdrgegl arvlcgeeqa dadagaqvcs lllaqsevrp
qclllvlanr teissklqlm kkhqsdlkkl gildfteqdv ashqsysqkt lialvtsgal
lavlgitgyf lmnrrswspt gerlelep
SEQ ID NO: 2
“MYLWLKLLAFGFAFLDTEVFVTGQSPTPSPTDAYLNASETTTLS
PSGSAVISTTTIATTPSKPTCDEKYANITVDYLYNKETKLFTAKLNVNENVECGNNTC
TNNEVHNLTECKNASVSISHNSCTAPDKTLILDVPPGVEKFQLHDCTQVEKADTTICL
KWKNIETFTCDTQNITYRFQCGNMIFDNKEIKLENLEPEHEYKCDSEILYNNHKFTNA
SKIIKTDFGSPGEPQIIFCRSEAAHQGVITWNPPQRSFHNFTLCYIKETEKDCLNLDK
NLIKYDLQNLKPYTKYVLSLHAYIIAKVQRNGSAAMCHFTTKSAPPSQVWNMTVSMTS
DNSMHVKCRPPRDRNGPHERYHLEVEAGNTLVRNESHKNCDFRVKDLQYSTDYTFKAY
FHNGDYPGEPFILHHSTSYNSKALIAFLAFLIIVTSIALLVVLYKIYDLHKKRSCNLD
EQQELVERDDEKQLMNVEPIHADILLETYKRKIADEGRLFLAEFQSIPRVFSKFPIKE
ARKPFNQNKNRYVDILPYDYNRVELSEINGDAGSNYINASYIDGFKEPRKYIAAQGPR
DETVDDFWRMIWEQKATVIVMVTRCEEGNRNKCAEYWPSMEEGTRAFGDVVVKINQHK
RCPDYIIQKLNIVNKKEKATGREVTHIQFTSWPDHGVPEDPHLLLKLRRRVNAFSNFF
SGPIVVHCSAGVGRTGTYIGIDAMLEGLEAENKVDVYGYVVKLRRQRCLMVQVEAQYI
LIHQALVEYNQFGETEVNLSELHPYLHNMKKRDPPSEPSPLEAEFQRLPSYRSWRTQH
IGNQEENKSKNRNSNVIPYDYNRVPLKHELEMSKESEHDSDESSDDDSDSEEPSKYIN
ASFIMSYWKPEVMIAAQGPLKETIGDFWQMIFQRKVKVIVMLTELKHGDQEICAQYWG
EGKQTYGDIEVDLKDTDKSSTYTLRVFELRHSKRKDSRTVYQYQYTNWSVEQLPAEPK
ELISMIQVVKQKLPQKNSSEGNKHHKSTPLLIHCRDGSQQTGIFCALLNLLESAETEE
VVDIFQVVKALRKARPGMVSTFEQYQFLYDVIASTYPAQNGQVKKNNHQEDKIEFDNE
VDKVKQDANCVNPLGAPEKLPEAKEQAEGSEPTSGTEGPEHSVNGPASPALNQGS”
SEQ ID NO: 3
MNLAISIALLLTVLQVSRGQKVTSLTACLVDQSLRLDCRHENTS
SSPIQYEFSLTRETKKHVLFGTVGVPEHTYRSRTNFTSKYNMKVLYLSAFTSKDEGTY
TCALHHSGHSPPISSQNVTVLRDKLVKCEGISLLAQNTSWLLLLLLSLSLLQATDFMS
L
SEQ ID NO: 4
MVLLWLTLLLIALPCLLQTKEDPNPPITNLRMKAKAQQLTWDLN
RNVTDIECVKDADYSMPAVNNSYCQFGAISLCEVTNYTVRVANPPFSTWILFPENSGK
PWAGAENLTCWIHDVDFLSCSWAVGPGAPADVQYDLYLNVANRRQQYECLHYKTDAQG
TRIGCRFDDISRLSSGSQSSHILVRGRSAAFGIPCTDKFVVFSQIEILTPPNMTAKCN
KTHSFMHWKMRSHFNRKFRYELQIQKRMQPVITEQVRDRTSFQLLNPGTYTVQIRARE
RVYEFLSAWSTPQRFECDQEEGANTRAWRTSLLIALGTLLALVCVFVICRRYLVMQRL
FPRIPHMKDPIGDSFQNDKLVVWEAGKAGLEECLVTEVQVVQKT
SEQ ID NO: 5
MANCEFSPVSGDKPCCRLSRRAQLCLGVSILVLILVVVLAVVVP
RWRQQWSGPGTTKRFPETVLARCVKYTEIHPEMRHVDCQSVWDAFKGAFISKHPCNIT
EEDYQPLMKLGTQTVPCNKILLWSRIKDLAHQFTQVQRDMFTLEDTLLGYLADDLTWC
GEFNTSKINYQSCPDWRKDCSNNPVSVFWKTVSRRFAEAACDVVHVMLNGSRSKIFDK
NSTFGSVEVHNLQPEKVQTLEAWVIHGGREDSRDLCQDPTIKELESIISKRNIQFSCK
NIYRPDKFLQCVKNPEDSSCTSEI
SEQ ID NO: 6
MPPPRLLFFLLFLTPMEVRPEEPLVVKVEEGDNAVLQCLKGTSD
GPTQQLTWSRESPLKPFLKLSLGLPGLGIHMRPLAIWLFIFNVSQQMGGFYLCQPGPP
SEKAWQPGWTVNVEGSGELFRWNVSDLGGLGCGLKNRSSEGPSSPSGKLMSPKLYVWA
KDRPEIWEGEPPCLPPRDSLNQSLSQDLTMAPGSTLWLSCGVPPDSVSRGPLSWTHVH
PKGPKSLLSLELKDDRPARDMWVMETGLLLPRATAQDAGKYYCHRGNLTMSFHLEITA
RPVLWHWLLRTGGWKVSAVTLAYLIFCLCSLVGILHLQRALVLRRKRKRMTDPTRRFF
KVTPPPGSGPQNQYGNVLSLPTPTSGLGRAQRWAAGLGGTAPSYGNPSSDVQADGALG
DSRSPPGVGPEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSYEDMRGILY
AAPQLRSIRGQPGPNHEEDADSYENMDNPDGPDPAWGGGGRMGTWSTR
SEQ ID NO: 7
WPLVAALLLGSACCGSAQLLFNKTKSVEFTFCNDTVVIPCFVT
NMEAQNTTEVYVKWKFKGRDIYTFDGALNKSTVPTDFSSAKIEVSQLLKGDASLKMDK
SDAVSHTGNYTCEVTELTREGETIIELKYRVVSWFSPNENILIVIFPIFAILLFWGQF
GIKTLKYRSGGMDEKTIALLVAGLVITVIVIVGAILFVPGEYSLKNATGLGLIVTSTG
ILILLHYYVFSTAIGLTSFVIAILVIQVIAYILAVVGLSLCIAACIPMHGPLLISGLS
ILALAQLLGLVYMKFVASNQKTIQPPRKAVEEPLNAFKESKGMMNDE”
SEQ ID NO: 8
MDYTLDLSVTTVTDYYYPDIFSSPCDAELIQTNGKLLLAVFYCL
LFVFSLLGNSLVILVLVVCKKLRSITDVYLLNLALSDLLFVFSFPFQTYYLLDQWVFG
TVMCKVVSGFYYIGFYSSMFFITLMSVDRYLAVVHAVYALKVRTIRMGTTLCLAVWLT
AIMATIPLLVFYQVASEDGVLQCYSFYNQQTLKWKIFTNFKMNILGLLIPFTIFMFCY
IKILHQLKRCQNHNKTKAIRLVLIVVIASLLFWVPFNVVLFLTSLHSMHILDGCSISQ
QLTYATHVTEIISFTHCCVNPVIYAFVGEKFKKHLSEIFQKSCSQIFNYLGRQMPRES
CEKSSSCQQHSSRSSSVDYIL
SEQ ID NO: 9
MSFPKAPLKRFNDPSGCAPSPGAYDVKTLEVLKGPVSFQKSQRF
KQQKESKQNLNVDKDTTLPASARKVKSSESKKESQKNDKDLKILEKEIRVLLQERGAQ
DRRIQDLETELEKMEARLNAALREKTSLSANNATLEKQLIELTRTNELLKSKFSENGN
QKNLRILSLELMKLRNKRETKMRGMMAKQEGMEMKLQVTQRSLEESQGKIAQLEGKLV
SIEKEKIDEKSETEKLLEYIEEISCASDQVEKYKLDIAQLEENLKEKNDEILSLKQSL
EENIVILSKQVEDLNVKCQLLEKEKEDHVNRNREHNENLNAEMQNLKQKFILEQQERE
KLQQKELQIDSLLQQEKELSSSLHQKLCSFQEEMVKEKNLFEEELKQTLDELDKLQQK
EEQAERLVKQLEEEAKSRAEELKLLEEKLKGKEAELEKSSAAHTQATLLLQEKYDSMV
QSLEDVTAQFESYKALTASEIEDLKLENSSLQEKAAKAGKNAEDVQHQILATESSNQE
YVRMLLDLQTKSALKETEIKEITVSFLQKITDLQNQLKQQEEDFRKQLEDEEGRKAEK
ENTTAELTEEINKWRLLYEELYNKTKPFQLQLDAFEVEKQALLNEHGAAQEQLNKIRD
SYAKLLGHQNLKQKIKHVVKLKDENSQLKSEVSKLRCQLAKKKQSETKLQEELNKVLG
IKHFDPSKAFHHESKENFALKTPLKEGNTNCYRAPMECQESWK
SEQ ID NO: 10
MDPQCTMGLSNILFVMAFLLSGAAPLKIQAYFNETADLPCQFAN
SQNQSLSELVVFWQDQENLVLNEVYLGKEKFDSVHSKYMGRTSFDSDSWTLRLHNLQI
KDKGLYQCIIHHKKPTGMIRIHQMNSELSVLANFSQPEIVPISNITENVYINLTCSSI
HGYPEPKKMSVLLRTKNSTIEYDGVMQKSQDNVTELYDVSISLSVSFPDVTSNMTIFC
ILETDKTRLLSSPFSIELEDPQPPPDHIPWITAVLPTVIICVMVFCLILWKWKKKKRP
RNSYKCGTNTMEREESEQTKKREKIHIPERSDEAQRVFKSSKTSSCDKSDTCF
SEQ ID NO: 11
atgctggtcc gcaggggcgc gcgcgcaggg cccaggatgc cgcggggctg gaccgcgctt
tgcttgctga gtttgctgcc ttctgggttc atgagtcttg acaacaacgg tactgctacc
ccagagttac ctacccaggg aacattttca aatgtttcta caaatgtatc ctaccaagaa
actacaacac ctagtaccct tggaagtacc agcctgcacc ctgtgtctca acatggcaat
gaggccacaa caaacatcac agaaacgaca gtcaaattca catctacctc tgtgataacc
tcagtttatg gaaacacaaa ctcttctgtc cagtcacaga cctctgtaat cagcacagtg
ttcaccaccc cagccaacgt ttcaactcca gagacaacct tgaagcctag cctgtcacct
ggaaatgttt cagacctttc aaccactagc actagccttg caacatctcc cactaaaccc
tatacatcat cttctcctat cctaagtgac atcaaggcag aaatcaaatg ttcaggcatc
agagaagtga aattgactca gggcatctgc ctggagcaaa ataagacctc cagctgtgcg
gagtttaaga aggacagggg agagggcctg gcccgagtgc tgtgtgggga ggagcaggct
gatgctgatg ctggggccca ggtatgctcc ctgctccttg cccagtctga ggtgaggcct
cagtgtctac tgctggtctt ggccaacaga acagaaattt ccagcaaact ccaacttatg
aaaaagcacc aatctgacct gaaaaagctg gggatcctag atttcactga gcaagatgtt
gcaagccacc agagctattc ccaaaagacc ctgattgcac tggtcacctc gggagccctg
ctggctgtct tgggcatcac tggctatttc ctgatgaatc gccgcagctg gagccccaca
ggagaaaggc tgggcgaaga cccttattac acggaaaacg gtggaggcca gggctatagc
tcaggacctg ggacctcccc tgaggctcag ggaaaggcca gtgtgaaccg aggggctcag
gaaaacggga ccggccaggc cacctccaga aacggccatt cagcaagaca acacgtggtg
gctgataccg aattgtga
SEQ ID NO: 12
atgtatttgt ggcttaaact cttggcattt ggctttgcct ttctggacac agaagtattt
gtgacagggc aaagcccaac accttccccc actggattga ctacagcaaa gatgcccagt
gttccacttt caagtgaccc cttacctact cacaccactg cattctcacc cgcaagcacc
tttgaaagag aaaatgactt ctcagagacc acaacttctc ttagtccaga caatacttcc
acccaagtat ccccggactc tttggataat gctagtgctt ttaataccac aggtgtttca
tcagtacaga cgcctcacct tcccacgcac gcagactcgc agacgccctc tgctggaact
gacacgcaga cattcagcgg ctccgccgcc aatgcaaaac tcaaccctac cccaggcagc
aatgctatct cagatgtccc aggagagagg agtacagcca gcacctttcc tacagaccca
gtttccccat tgacaaccac cctcagcctt gcacaccaca gctctgctgc cttacctgca
cgcacctcca acaccaccat cacagcgaac acctcagatg cctaccttaa tgcctctgaa
acaaccactc tgagcccttc tggaagcgct gtcatttcaa ccacaacaat agctactact
ccatctaagc caacatgtga tgaaaaatat gcaaacatca ctgtggatta cttatataac
aaggaaacta aattatttac agcaaagcta aatgttaatg agaatgtgga atgtggaaac
aatacttgca caaacaatga ggtgcataac cttacagaat gtaaaaatgc gtctgtttcc
atatctcata attcatgtac tgctcctgat aagacattaa tattagatgt gccaccaggg
gttgaaaagt ttcagttaca tgattgtaca caagttgaaa aagcagatac tactatttgt
ttaaaatgga aaaatattga aacctttact tgtgatacac agaatattac ctacagattt
cagtgtggta atatgatatt tgataataaa gaaattaaat tagaaaacct tgaacccgaa
catgagtata agtgtgactc agaaatactc tataataacc acaagtttac taacgcaagt
aaaattatta aaacagattt tgggagtcca ggagagcctc agattatttt ttgtagaagt
gaagctgcac atcaaggagt aattacctgg aatccccctc aaagatcatt tcataatttt
accctctgtt atataaaaga gacagaaaaa gattgcctca atctggataa aaacctgatc
aaatatgatt tgcaaaattt aaaaccttat acgaaatatg ttttatcatt acatgcctac
atcattgcaa aagtgcaacg taatggaagt gctgcaatgt gtcatttcac aactaaaagt
gctcctccaa gccaggtctg gaacatgact gtctccatga catcagataa tagtatgcat
gtcaagtgta ggcctcccag ggaccgtaat ggcccccatg aacgttacca tttggaagtt
gaagctggaa atactctggt tagaaatgag tcgcataaga attgcgattt ccgtgtaaaa
gatcttcaat attcaacaga ctacactttt aaggcctatt ttcacaatgg agactatcct
ggagaaccct ttattttaca tcattcaaca tcttataatt ctaaggcact gatagcattt
ctggcatttc tgattattgt gacatcaata gccctgcttg ttgttctcta caaaatctat
gatctacata agaaaagatc ctgcaattta gatgaacagc aggagcttgt tgaaagggat
gatgaaaaac aactgatgaa tgtggagcca atccatgcag atattttgtt ggaaacttat
aagaggaaga ttgctgatga aggaagactt tttctggctg aatttcagag catcccgcgg
gtgttcagca agtttcctat aaaggaagct cgaaagccct ttaaccagaa taaaaaccgt
tatgttgaca ttcttcctta tgattataac cgtgttgaac tctctgagat aaacggagat
gcagggtcaa actacataaa tgccagctat attgatggtt tcaaagaacc caggaaatac
attgctgcac aaggtcccag ggatgaaact gttgatgatt tctggaggat gatttgggaa
cagaaagcca cagttattgt catggtcact cgatgtgaag aaggaaacag gaacaagtgt
gcagaatact ggccgtcaat ggaagagggc actcgggctt ttggagatgt tgttgtaaag
atcaaccagc acaaaagatg tccagattac atcattcaga aattgaacat tgtaaataaa
aaagaaaaag caactggaag agaggtgact cacattcagt tcaccagctg gccagaccac
ggggtgcctg aggatcctca cttgctcctc aaactgagaa ggagagtgaa tgccttcagc
aatttcttca gtggtcccat tgtggtgcac tgcagtgctg gtgttgggcg cacaggaacc
tatatcggaa ttgatgccat gctagaaggc ctggaagccg agaacaaagt ggatgtttat
ggttatgttg tcaagctaag gcgacagaga tgcctgatgg ttcaagtaga ggcccagtac
atcttgatcc atcaggcttt ggtggaatac aatcagtttg gagaaacaga agtgaatttg
tctgaattac atccatatct acataacatg aagaaaaggg atccacccag tgagccgtct
ccactagagg ctgaattcca gagacttcct tcatatagga gctggaggac acagcacatt
ggaaatcaag aagaaaataa aagtaaaaac aggaattcta atgtcatccc atatgactat
aacagagtgc cacttaaaca tgagctggaa atgagtaaag agagtgagca tgattcagat
gaatcctctg atgatgacag tgattcagag gaaccaagca aatacatcaa tgcatctttt
ataatgagct actggaaacc tgaagtgatg attgctgctc agggaccact gaaggagacc
attggtgact tttggcagat gatcttccaa agaaaagtca aagttattgt tatgctgaca
gaactgaaac atggagacca ggaaatctgt gctcagtact ggggagaagg aaagcaaaca
tatggagata ttgaagttga cctgaaagac acagacaaat cttcaactta tacccttcgt
gtctttgaac tgagacattc caagaggaaa gactctcgaa ctgtgtacca gtaccaatat
acaaactgga gtgtggagca gcttcctgca gaacccaagg aattaatctc tatgattcag
gtcgtcaaac aaaaacttcc ccagaagaat tcctctgaag ggaacaagca tcacaagagt
acacctctac tcattcactg cagggatgga tctcagcaaa cgggaatatt ttgtgctttg
ttaaatctct tagaaagtgc ggaaacagaa gaggtagtgg atatttttca agtggtaaaa
gctctacgca aagctaggcc aggcatggtt tccacattcg agcaatatca attcctatat
gacgtcattg ccagcaccta ccctgctcag aatggacaag taaagaaaaa caaccatcaa
gaagataaaa ttgaatttga taatgaagtg gacaaagtaa agcaggatgc taattgtgtt
aatccacttg gtgccccaga aaagctccct gaagcaaagg aacaggctga aggttctgaa
cccacgagtg gcactgaggg gccagaacat tctgtcaatg gtcctgcaag tccagcttta
aatcaaggtt catag
SEQ ID NO: 13
atgaacctgg ccatcagcat cgctctcctg ctaacagtct tgcaggtctc ccgagggcag
aaggtgacca gcctaacggc ctgcctagtg gaccagagcc ttcgtctgga ctgccgccat
gagaatacca gcagttcacc catccagtac gagttcagcc tgacccgtga gacaaagaag
cacgtgctct ttggcactgt gggggtgcct gagcacacat accgctcccg aaccaacttc
accagcaaat acaacatgaa ggtcctctac ttatccgcct tcactagcaa ggacgagggc
acctacacgt gtgcactcca ccactctggc cattccccac ccatctcctc ccagaacgtc
acagtgctca gagacaaact ggtcaagtgt gagggcatca gcctgctggc tcagaacacc
tcgtggctgc tgctgctcct gctctccctc tccctcctcc aggccacgga tttcatgtcc
ctgtga
SEQ ID NO: 14
atggtcctcc tttggctcac gctgctcctg atcgccctgc cctgtctcct gcaaacgaag
gaagatccaa acccaccaat cacgaaccta aggatgaaag caaaggctca gcagttgacc
tgggacctta acagaaatgt gaccgatatc gagtgtgtta aagacgccga ctattctatg
ccggcagtga acaatagcta ttgccagttt ggagcaattt ccttatgtga agtgaccaac
tacaccgtcc gagtggccaa cccaccattc tccacgtgga tcctcttccc tgagaacagt
gggaagcctt gggcaggtgc ggagaatctg acctgctgga ttcatgacgt ggatttcttg
agctgcagct gggcggtagg cccgggggcc cccgcggacg tccagtacga cctgtacttg
aacgttgcca acaggcgtca acagtacgag tgtcttcact acaaaacgga tgctcaggga
acacgtatcg ggtgtcgttt cgatgacatc tctcgactct ccagcggttc tcaaagttcc
cacatcctgg tgcggggcag gagcgcagcc ttcggtatcc cctgcacaga taagtttgtc
gtcttttcac agattgagat attaactcca cccaacatga ctgcaaagtg taataagaca
cattccttta tgcactggaa aatgagaagt catttcaatc gcaaatttcg ctatgagctt
cagatacaaa agagaatgca gcctgtaatc acagaacagg tcagagacag aacctccttc
cagctactca atcctggaac gtacacagta caaataagag cccgggaaag agtgtatgaa
ttcttgagcg cctggagcac cccccagcgc ttcgagtgcg accaggagga gggcgcaaac
acacgtgcct ggcggacgtc gctgctgatc gcgctgggga cgctgctggc cctggtctgt
gtcttcgtga tctgcagaag gtatctggtg atgcagagac tctttccccg catccctcac
atgaaagacc ccatcggtga cagcttccaa aacgacaagc tggtggtctg ggaggcgggc
aaagccggcc tggaggagtg tctggtgact gaagtacagg tcgtgcagaa aacttga
SEQ ID NO: 15
atggccaact gcgagttcag cccggtgtcc ggggacaaac cctgctgccg gctctctagg
agagcccaac tctgtcttgg cgtcagtatc ctggtcctga tcctcgtcgt ggtgctcgcg
gtggtcgtcc cgaggtggcg ccagcagtgg agcggtccgg gcaccaccaa gcgctttccc
gagaccgtcc tggcgcgatg cgtcaagtac actgaaattc atcctgagat gagacatgta
gactgccaaa gtgtatggga tgctttcaag ggtgcattta tttcaaaaca tccttgcaac
attactgaag aagactatca gccactaatg aagttgggaa ctcagaccgt accttgcaac
aagattcttc tttggagcag aataaaagat ctggcccatc agttcacaca ggtccagcgg
gacatgttca ccctggagga cacgctgcta ggctaccttg ctgatgacct cacatggtgt
ggtgaattca acacttccaa aataaactat caatcttgcc cagactggag aaaggactgc
agcaacaacc ctgtttcagt attctggaaa acggtttccc gcaggtttgc agaagctgcc
tgtgatgtgg tccatgtgat gctcaatgga tcccgcagta aaatctttga caaaaacagc
acttttggga gtgtggaagt ccataatttg caaccagaga aggttcagac actagaggcc
tgggtgatac atggtggaag agaagattcc agagacttat gccaggatcc caccataaaa
gagctggaat cgattataag caaaaggaat attcaatttt cctgcaagaa tatctacaga
cctgacaagt ttcttcagtg tgtgaaaaat cctgaggatt catcttgcac atctgagatc
tga
SEQ ID NO: 16
atgccacctc ctcgcctcct cttcttcctc ctcttcctca cccccatgga agtcaggccc
gaggaacctc tagtggtgaa ggtggaagag ggagataacg ctgtgctgca gtgcctcaag
gggacctcag atggccccac tcagcagctg acctggtctc gggagtcccc gcttaaaccc
ttcttaaaac tcagcctggg gctgccaggc ctgggaatcc acatgaggcc cctggccatc
tggcttttca tcttcaacgt ctctcaacag atggggggct tctacctgtg ccagccgggg
cccccctctg agaaggcctg gcagcctggc tggacagtca atgtggaggg cagcggggag
ctgttccggt ggaatgtttc ggacctaggt ggcctgggct gtggcctgaa gaacaggtcc
tcagagggcc ccagctcccc ttccgggaag ctcatgagcc ccaagctgta tgtgtgggcc
aaagaccgcc ctgagatctg ggagggagag cctccgtgtc tcccaccgag ggacagcctg
aaccagagcc tcagccagga cctcaccatg gcccctggct ccacactctg gctgtcctgt
ggggtacccc ctgactctgt gtccaggggc cccctctcct ggacccatgt gcaccccaag
gggcctaagt cattgctgag cctagagctg aaggacgatc gcccggccag agatatgtgg
gtaatggaga cgggtctgtt gttgccccgg gccacagctc aagacgctgg aaagtattat
tgtcaccgtg gcaacctgac catgtcattc cacctggaga tcactgctcg gccagtacta
tggcactggc tgctgaggac tggtggctgg aaggtctcag ctgtgacttt ggcttatctg
atcttctgcc tgtgttccct tgtgggcatt cttcatcttc aaagagccct ggtcctgagg
aggaaaagaa agcgaatgac tgaccccacc aggagattct tcaaagtgac gcctccccca
ggaagcgggc cccagaacca gtacgggaac gtgctgtctc tccccacacc cacctcaggc
ctcggacgcg cccagcgttg ggccgcaggc ctggggggca ctgccccgtc ttatggaaac
ccgagcagcg acgtccaggc ggatggagcc ttggggtccc ggagcccgcc gggagtgggc
ccagaagaag aggaagggga gggctatgag gaacctgaca gtgaggagga ctccgagttc
tatgagaacg actccaacct tgggcaggac cagctctccc aggatggcag cggctacgag
aaccctgagg atgagcccct gggtcctgag gatgaagact ccttctccaa cgctgagtct
tatgagaacg aggatgaaga gctgacccag ccggtcgcca ggacaatgga cttcctgagc
cctcatgggt cagcctggga ccccagccgg gaagcaacct ccctggggtc ccagtcctat
gaggatatga gaggaatcct gtatgcagcc ccccagctcc gctccattcg gggccagcct
ggacccaatc atgaggaaga tgcagactct tatgagaaca tggataatcc cgatgggcca
gacccagcct ggggaggagg gggccgcatg ggcacctgga gcaccaggtg a
SEQ ID NO: 17
atgtggcccc tggtagcggc gctgttgctg ggctcggcgt gctgcggatc agctcagcta
ctatttaata aaacaaaatc tgtagaattc acgttttgta atgacactgt cgtcattcca
tgctttgtta ctaatatgga ggcacaaaac actactgaag tatacgtaaa gtggaaattt
aaaggaagag atatttacac ctttgatgga gctctaaaca agtccactgt ccccactgac
tttagtagtg caaaaattga agtctcacaa ttactaaaag gagatgcctc tttgaagatg
gataagagtg atgctgtctc acacacagga aactacactt gtgaagtaac agaattaacc
agagaaggtg aaacgatcat cgagctaaaa tatcgtgttg tttcatggtt ttctccaaat
gaaaatattc ttattgttat tttcccaatt tttgctatac tcctgttctg gggacagttt
ggtattaaaa cacttaaata tagatccggt ggtatggatg agaaaacaat tgctttactt
gttgctggac tagtgatcac tgtcattgtc attgttggag ccattctttt cgtcccaggt
gaatattcat taaagaatgc tactggcctt ggtttaattg tgacttctac agggatatta
atattacttc actactatgt gtttagtaca gcgattggat taacctcctt cgtcattgcc
atattggtta ttcaggtgat agcctatatc ctcgctgtgg ttggactgag tctctgtatt
gcggcgtgta taccaatgca tggccctctt ctgatttcag gtttgagtat cttagctcta
gcacaattac ttggactagt ttatatgaaa tttgtggctt ccaatcagaa gactatacaa
cctcctagga aagctgtaga ggaacccctt aatgcattca aagaatcaaa aggaatgatg
aatgatgaat aa
SEQ ID NO: 18
tttgtagtgg gaggatacct ccagagaggc tgctgctcat tgagctgcac tcacatgagg
atacagactt tgtgaagaag gaattggcaa cactgaaacc tccagaacaa aggctgtcac
taaggtcccg ctgccttgat ggattataca cttgacctca gtgtgacaac agtgaccgac
tactactacc ctgatatctt ctcaagcccc tgtgatgcgg aacttattca gacaaatggc
aagttgctcc ttgctgtctt ttattgcctc ctgtttgtat tcagtcttct gggaaacagc
ctggtcatcc tggtccttgt ggtctgcaag aagctgagga gcatcacaga tgtatacctc
ttgaacctgg ccctgtctga cctgcttttt gtcttctcct tcccctttca gacctactat
ctgctggacc agtgggtgtt tgggactgta atgtgcaaag tggtgtctgg cttttattac
attggcttct acagcagcat gtttttcatc accctcatga gtgtggacag gtacctggct
gttgtccatg ccgtgtatgc cctaaaggtg aggacgatca ggatgggcac aacgctgtgc
ctggcagtat ggctaaccgc cattatggct accatcccat tgctagtgtt ttaccaagtg
gcctctgaag atggtgttct acagtgttat tcattttaca atcaacagac tttgaagtgg
aagatcttca ccaacttcaa aatgaacatt ttaggcttgt tgatcccatt caccatcttt
atgttctgct acattaaaat cctgcaccag ctgaagaggt gtcaaaacca caacaagacc
aaggccatca ggttggtgct cattgtggtc attgcatctt tacttttctg ggtcccattc
aacgtggttc ttttcctcac ttccttgcac agtatgcaca tcttggatgg atgtagcata
agccaacagc tgacttatgc cacccatgtc acagaaatca tttcctttac tcactgctgt
gtgaaccctg ttatctatgc ttttgttggg gagaagttca agaaacacct ctcagaaata
tttcagaaaa gttgcagcca aatcttcaac tacctaggaa gacaaatgcc tagggagagc
tgtgaaaagt catcatcctg ccagcagcac tcctcccgtt cctccagcgt agactacatt
ttgtgaggat caatgaagac taaatataaa aaacattttc ttgaatggca tgctagtagc
agtgagcaaa ggtgtgggtg tgaaaggttt ccaaaaaaag ttcagcatga aggatgccat
atatgttgtt gccaacactt ggaacacaat gactaaagac atagttgtgc atgcctggca
caacatcaag cctgtgattg tgtttattga tgatgttgaa caagtggtaa ctttaaagga
ttctgtatgc caagtgaaaa aaaaagatgt ctgacctcct tacatat
SEQ ID NO: 19
attctttctt cgtgttcctg tgcgggattg gtgtgcccag gggtttggct ttccaattgg
ctaacgccgg ggtgggtggg gaatgtgggg agatttgaat ttgaaaccgg tagggagtga
taatccgcat tcagttgtcg aggagtgcca gtcaccttca gtttctggag ctggccgtca
acatgtcctt tcctaaggcg cccttgaaac gattcaatga cccttctggt tgtgcaccat
ctccaggtgc ttatgatgtt aaaactttag aagtattgaa aggaccagta tcctttcaga
aatcacaaag atttaaacaa caaaaagaat ctaaacaaaa tcttaatgtt gacaaagata
ctaccttgcc tgcttcagct agaaaagtta agtcttcgga atcaaagaag gaatctcaaa
agaatgataa agatttgaag atattagaga aagagattcg tgttcttcta caggaacgtg
gtgcccagga caggcggatc caggatctgg aaactgagtt ggaaaagatg gaagcaaggc
taaatgctgc actaagggaa aaaacatctc tctctgcaaa taatgctaca ctggaaaaac
aacttattga attgaccagg actaatgaac tactaaaatc taagttttct gaaaatggta
accagaagaa tttgagaatt ctaagcttgg agttgatgaa acttagaaac aaaagagaaa
caaagatgag gggtatgatg gctaagcaag aaggcatgga gatgaagctg caggtcaccc
aaaggagtct cgaagagtct caagggaaaa tagcccaact ggagggaaaa cttgtttcaa
tagagaaaga aaagattgat gaaaaatctg aaacagaaaa actcttggaa tacatcgaag
aaattagttg tgcttcagat caagtggaaa aatacaagct agatattgcc cagttagaag
aaaatttgaa agagaagaat gatgaaattt taagccttaa gcagtctctt gaggagaata
ttgttatatt atctaaacaa gtagaagatc taaatgtgaa atgtcagctg cttgaaaaag
aaaaagaaga ccatgtcaac aggaatagag aacacaacga aaatctaaat gcagagatgc
aaaacttaaa acagaagttt attcttgaac aacaggaacg tgaaaagctt caacaaaaag
aattacaaat tgattcactt ctgcaacaag agaaagaatt atcttcgagt cttcatcaga
agctctgttc ttttcaagag gaaatggtta aagagaagaa tctgtttgag gaagaattaa
agcaaacact ggatgagctt gataaattac agcaaaagga ggaacaagct gaaaggctgg
tcaagcaatt ggaagaggaa gcaaaatcta gagctgaaga attaaaactc ctagaagaaa
agctgaaagg gaaggaggct gaactggaga aaagtagtgc tgctcatacc caggccaccc
tgcttttgca ggaaaagtat gacagtatgg tgcaaagcct tgaagatgtt actgctcaat
ttgaaagcta taaagcgtta acagccagtg agatagaaga tcttaagctg gagaactcat
cattacagga aaaagcggcc aaggctggga aaaatgcaga ggatgttcag catcagattt
tggcaactga gagctcaaat caagaatatg taaggatgct tctagatctg cagaccaagt
cagcactaaa ggaaacagaa attaaagaaa tcacagtttc ttttcttcaa aaaataactg
atttgcagaa ccaactcaag caacaggagg aagactttag aaaacagctg gaagatgaag
aaggaagaaa agctgaaaaa gaaaatacaa cagcagaatt aactgaagaa attaacaagt
ggcgtctcct ctatgaagaa ctatataata aaacaaaacc ttttcagcta caactagatg
cttttgaagt agaaaaacag gcattgttga atgaacatgg tgcagctcag gaacagctaa
ataaaataag agattcatat gctaaattat tgggtcatca gaatttgaaa caaaaaatca
agcatgttgt gaagttgaaa gatgaaaata gccaactcaa atcggaagta tcaaaactcc
gctgtcagct tgctaaaaaa aaacaaagtg agacaaaact tcaagaggaa ttgaataaag
ttctaggtat caaacacttt gatccttcaa aggcttttca tcatgaaagt aaagaaaatt
ttgccctgaa gaccccatta aaagaaggca atacaaactg ttaccgagct cctatggagt
gtcaagaatc atggaagtaa acatctgaga aacctgttga agattatttc attcgtcttg
ttgttattga tgttgctgtt attatatttg acatgggtat tttataatgt tgtatttaat
tttaactgcc aatccttaaa tatgtgaaag gaacattttt taccaaagtg tcttttgaca
ttttattttt tcttgcaaat acctcctccc taatgctcac ctttatcacc tcattctgaa
ccctttcgct ggctttccag cttagaatgc atctcatcaa cttaaaagtc agtatcatat
tattatcctc ctgttctgaa accttagttt caagagtcta aaccccagat tcttcagctt
gatcctggag gtcttttcta gtctgagctt ctttagctag gctaaaacac cttggcttgt
tattgcctct actttgattc tgataatgct cacttggtcc tacctattat ccttctactt
gtccagttca aataagaaat aaggacaagc ctaacttcat agaaacctct ctatttttaa
tcagttgttt aataatttac aggttcttag gctccatcct gtttgtatga aattataatc
tgtggattgg cctttaagcc tgcattctta acaaactctt cagttaattc ttagatacac
taaaaatctg agaaactcta catgtaacta tttcttcaga gtttgtcata tactgcttgt
catctgcatg tctactcagc atttgattaa catttgtgta atatgaaata aaattacaca
gtaagtcatt taaccaatta aaaa
SEQ ID NO: 20
ggaaggcttg cacagggtga aagctttgct tctctgctgc tgtaacaggg actagcacag
acacacggat gagtggggtc atttccagat attaggtcac agcagaagca gccaaaatgg
atccccagtg cactatggga ctgagtaaca ttctctttgt gatggccttc ctgctctctg
gtgctgctcc tctgaagatt caagcttatt tcaatgagac tgcagacctg ccatgccaat
ttgcaaactc tcaaaaccaa agcctgagtg agctagtagt attttggcag gaccaggaaa
acttggttct gaatgaggta tacttaggca aagagaaatt tgacagtgtt cattccaagt
atatgggccg cacaagtttt gattcggaca gttggaccct gagacttcac aatcttcaga
tcaaggacaa gggcttgtat caatgtatca tccatcacaa aaagcccaca ggaatgattc
gcatccacca gatgaattct gaactgtcag tgcttgctaa cttcagtcaa cctgaaatag
taccaatttc taatataaca gaaaatgtgt acataaattt gacctgctca tctatacacg
gttacccaga acctaagaag atgagtgttt tgctaagaac caagaattca actatcgagt
atgatggtgt tatgcagaaa tctcaagata atgtcacaga actgtacgac gtttccatca
gcttgtctgt ttcattccct gatgttacga gcaatatgac catcttctgt attctggaaa
ctgacaagac gcggctttta tcttcacctt tctctataga gcttgaggac cctcagcctc
ccccagacca cattccttgg attacagctg tacttccaac agttattata tgtgtgatgg
ttttctgtct aattctatgg aaatggaaga agaagaagcg gcctcgcaac tcttataaat
gtggaaccaa cacaatggag agggaagaga gtgaacagac caagaaaaga gaaaaaatcc
atatacctga aagatctgat gaagcccagc gtgtttttaa aagttcgaag acatcttcat
gcgacaaaag tgatacatgt ttttaattaa agagtaaagc ccatacaagt attcattttt
tctacccttt cctttgtaag ttcctgggca acctttttga tttcttccag aaggcaaaaa
gacattacca tgagtaataa gggggctcca ggactccctc taagtggaat agcctccctg
taactccagc tctgctccgt atgccaagag gagactttaa ttctcttact gcttcttttc
acttcagagc acacttatgg gccaagccca gcttaatggc tcatgacctg gaaataaaat
ttaggaccaa tacctcctcc agatcagatt cttctcttaa tttcatagat tgtgtttttt
ttttaaatag acctctcaat ttctggaaaa ctgcctttta tctgcccaga attctaagct
ggtgccccac tgaattttgt gtgtacctgt gactaaacaa ctacctcctc agtctgggtg
ggacttatgt atttatgacc ttatagtgtt aatatcttga aacatagaga tctatgtact
gtaatagtgt gattactatg ctctagagaa aagtctaccc ctgctaagga gttctcatcc
ctctgtcagg gtcagtaagg aaaacggtgg cctagggtac aggcaacaat gagcagacca
acctaaattt ggggaaatta ggagaggcag agatagaacc tggagccact tctatctggg
ctgttgctaa tattgaggag gcttgcccca cccaacaagc catagtggag agaactgaat
aaacaggaaa atgccagagc ttgtgaaccc tgtttctctt gaagaactga ctagtgagat
ggcctgggga agctgtgaaa gaaccaaaag agatcacaat actcaaaaga gagagagaga
gaaaaaagag agatcttgat ccacagaaat acatgaaatg tctggtctgt ccaccccatc
aacaagtctt gaaacaagca acagatggat agtctgtcca aatggacata agacagacag
cagtttccct ggtggtcagg gaggggtttt ggtgataccc aagttattgg gatgtcatct
tcctggaagc agagctgggg agggagagcc atcaccttga taatgggatg aatggaagga
ggcttaggac tttccactcc tggctgagag aggaagagct gcaacggaat taggaagacc
aagacacaga tcacccgggg cttacttagc ctacagatgt cctacgggaa cgtgggctgg
cccagcatag ggctagcaaa tttgagttgg atgattgttt ttgctcaagg caaccagagg
aaacttgcat acagagacag atatactggg agaaatgact ttgaaaacct ggctctaagg
tgggatcact aagggatggg gcagtctctg cccaaacata aagagaactc tggggagcct
gagccacaaa aatgttcctt tattttatgt aaaccctcaa gggttataga ctgccatgct
agacaagctt gtccatgtaa tattcccatg tttttaccct gcccctgcct tgattagact
cctagcacct ggctagtttc taacatgttt tgtgcagcac agtttttaat aaatgcttgt
tacattcaaa aaaaaaaaaa aaaaaaaaaa aaaaaaaaaa aaaaaaaaaa aaaaaaaaaa
aaaaaaaaaa aaaaaaaaaa aaaa

### EXAMPLES

### Example 1

### AML Diagnostic FACS Screen

### Patient Samples

Bone marrow samples from AML patients and patients undergoing orthopaedic surgery with normal blood counts/films were obtained with informed consent (protocols 06/Q1606/110 and 05/MRE07/74). Bone marrow samples were received in EDTA (Heparin is a suitable alternative) and were lysed with ammonium chloride prior to immunostaining.

Samples received in culture medium were washed and the sample resuspended in PBS before analysis. To do this the sample was transferred to a conical tube (10 ml), which was topped up with PBS and centrifuged for 5 minutes at 3000 rpm. The supernatant was removed using a pipette and the cells resuspended in PBS to ˜2 ml.

Occasionally diagnostic panels were run on peripheral blood samples if the blast count was high enough. The technique used was identical to that for bone marrow.

Samples were prepared as soon as possible after receipt.

### Lysis

Ammonium Chloride was used for lysis:

### NH₄CL₂: 8.30 g

### KHCO₃: 1.00 g

### Na₂EDTa: 0.38 g

Made up to 1 Litre with Distilled Water for a working solution which was stable for 1 month.
1. Diluted BM/PB with a least a 10 fold excess of Ammonium Chloride Lyse, in an appropriate centrifuge tube (20 or 50 ml conical tubes)
2. Incubated for 5-10 minutes, examined for red cell lysis. If red cells showed resistance to lysis incubated for a further 5-10 minutes at 37 or 4° C.
3. Spun sample down at 300 g for 5 min
4. Tipped off supernatant and topped up with PBS
5. Spun sample down at 300 g for 5 min
6. Resuspended pellet in 1 ml of PBS and obtained white cell count

Adjusted the white cell count of samples for diagnostic studies to ˜5×10⁹/l by dilution with PBS.

Red cell contamination will interfere with scatter plots and reduce overall MRD %

Excessive lysis will result in changes to FSC/SSC scatter properties

### Panel Set Up

1. Added 5 ul of the following antibodies to a FACS tube:
CD34 Percp Becton Dickinson 345803
CD45 APC-H7 Becton Dickinson 641389
CD47 PE Pharmingen 556046
CD38 PE-CY7 Becton Dickinson 335825
CD19 Pacific Bleu E Biosciences 87-0199-33
CD90
CD123
CRR8
RHAMM
CD86
2. Added 100 μl of pre lysed sample to each of tubes
3. Vortexed each tube
4. Incubated at RT in dark for 15 minutes
5. Topped up each tube with PBS
6. Centrifuged at 300 g for 5 minutes
7. Tipped off/aspirate supernatant
8. Resuspended cell pellet in 5001 PBS
9. Vortexed well
10. The samples were now ready for acquisition

### Acquisition

1. Acquired 500,000 cells using a FacsDiva Protocol as follows:
2. Define a CD45 vs. SSC plot
3. Gate all CD45 positive cells (Gate P1)
4. Project these cells onto a CD34 vs. SSC plot
5. Gate CD34 positive events (Gate P2)
6. Refine the CD34 positive population on a CD45 vs. SSC plot (Gate P3).
7. Further refine the CD34 population on a FSC vs. SSC plot (Gate P4)
8. The combined information satisfying gates P1, 2, 3 and 4 are projected on a CD19 vs. CD34 plot. CD34+CD19− cells are gated (Gate P5)
9. Events from P5 (CD34+CD19−) are projected onto a CD45 vs. CD34 gate.
10. Events from P5 (CD34+CD19−) are projected onto an exponential CD38 vs. CD34 gate.
11. Define regions for CD38−CD34+ (gate P7) and CD38−CD34+ cells (gate P8)
12. Events from P5 (CD34+CD19−) are projected onto a CD38 vs. CD47 plot.

### Results

### Immunophenotype of Lin-CD34+CD38− and Lin-CD34+CD38+ Compartments in Human AML: There are Two Major Immunophenotypic Groups of AML.

We used cell surface markers to compare patterns of stem/progenitor-cell immunophenotypes in CD34+ primary AML and normal control samples. The immunophenotypic gating strategy is illustrated in FIG. 1 was validated by reanalyzing purity of FACS-sorted stem/progenitor populations and showed that the sorted populations were >99% pure (FIG. 2). In vitro colony assays and in vivo assays confirmed the functional potential of sorted normal stem/progenitor cells. Using this approach, we immunophenotyped 82 primary AML samples (57 de novo AML, 14 secondary AML, 6 relapsed AML and 5 refractory AML—spanning a range of FAB subtypes, cytogenetic categories and FLT3 and NPM1 mutation states and 8 age-matched control marrow samples.

For the first time, we showed that there are two major immunophenotypic groups in primary human CD34+ AML with respect to these markers (FIG. 3). 82% of the 56 AML samples (and 77.8% of the 45 de novo AML samples), most of the Lin-CD34+CD38−cells are CD34+CD38−CD90−CD45RA+ (hereafter termed “CD38−CD45RA+”) (57%-100% of CD34+CD38− cells and in most cases >90%). Hereafter, this group is named “CD45RA+ expanded” group (FIG. 3A ii). Less commonly, in 22.2% of the 56 primary AML samples there is dominant Lin-CD34+CD38−CD90−CD45RA− population (66%-99% of CD34+CD38− cells and in most cases >90%). This group was only seen in de novo AML cases. Hereafter, this group is named “MPP-like expanded” group (FIG. 3A iii).

Within these two major groups there are variations of CD38 expression between AML samples (FIG. 3B). Within the both groups nearly all cells can be CD38− (FIG. 3B ii and iv), or a proportion of cells can express CD38 (FIG. 3B iii and v) and finally in the 45RA+-like expanded group the majority of cells can be CD38+. The central novel finding is that 82% of a broad range of primary CD34+ expressing AML samples have one major distinct CD38−CD45RA+ population within the CD34+CD38− compartment and a corresponding GMP-like population within the CD34+CD38+ compartment.

### Leukaemic Stem Cell Hierarchy in CD45RA+ Expanded AML

Given the CD45RA+ expanded group is the major group, we focused on dissecting which populations within this group had LSC activity in a xenotransplant assay. Previous data had shown that in CD34+ AML LSC activity resides in the CD34 compartment. Therefore, FACS-sorted CD38−CD45RA+ and GMP-like cells from 6 patients (10 populations in total) were injected intravenously into NOD-SCID mice treated with anti-CD122 antibody to remove residual NK-cells (FIG. 4A). Samples details are in FIG. 12. 105 cells/mouse of each population were injected into 4 mice. Cells from 5 out of 6 patients were detected in bone marrow in all 4 mice injected for each of the 10 populations from these 5 patients. There was a similar level of engraftment from CD38−CD45RA+ and GMP-like populations, consistent with LSC activity in both the CD38− and CD38+ compartments. FISH analysis on FACS-sorted human engrafted cells from mice injected with CD38−CD45RA+ and GMP-like cells from 2 AML patients with cytogenetic abnormalities showed that nearly all cells were leukaemic (FIG. 4B and FIG. 13).

FACS analysis confirmed that for both CD38−CD45RA+ and GMP-like injected populations, CD34+ and CD34− cells were detected in bone marrow (FIG. 4Ci and iv). In both CD34+ and CD34− populations in mice injected with CD38−CD45RA+ (FIG. 4C ii and iii) and GMP-like (FIG. 4Cv and vi) populations, nearly all cells expressed CD33 but not CD19, consistent with leukaemic myeloid-restricted engraftment as opposed to multi-potential myelo-lymphoid engraftment from normal blood cells. In mice injected with CD38−CD45RA+ cells, Lin-CD34+CD38−CD90−CD45RA+ cells were detected consistent with self-renewal of this population (FIG. 4Di and ii). CD38−CD45RA+ cells also gave rise to GMP-like cells in vivo (FIG. 4D iii). In contrast, when GMP-like cells are injected in mice, <0.1% of the CD34+ cells are CD34+CD38−CD45RA+ (FIG. 4D iv). Taken together, this is consistent with an in vivo hierarchy where a minority of CD34+CD38− cells self renew and give rise to GMP-like cells whereas GMP-like cells cannot give rise to CD34+CD38−CD45RA+ cells.

To prove that CD38−CD45RA+ and GMP-like AML populations have leukemic stem cell activity defined by secondary engraftment, human cells were harvested from primary recipients and injected into secondary NOD-SCID hosts treated with anti-CD122 antibody (FIG. 4E). Engraftment in secondary hosts was seen at 12 weeks from all 5 samples with no difference in engraftment level between cells taken from primary hosts transplanted with either CD38−CD45RA+ or GMP-like populations. The overall engraftment level of was lower in secondary hosts compared to primary transplanted animals as previously reported in the literature. Detailed immunophenotypic analysis of the engrafted cells showed that in all cases >99% of the human cells were CD33+ and CD19− consistent with engraftment of myeloid LSC rather than normal HSC (FIG. 5). The majority of the hCD34+ cells were CD38+CD110+CD45RA+ (i.e. GMP-like) regardless of whether the injected cells were from animals initially injected with CD38−CD45RA+ or GMP-like LSC. This would be consistent with differentiation of CD38−CD45RA+ cells into GMP-like cells but not in the reverse direction. In summary, both CD38−CD45RA+ and GMP-like populations, within the same patient, have LSC activity. In vivo, CD38−CD45RA+ LSC give rise to cells with a GMP-like phenotype and the converse does not occur.

### In Vitro Differentiation of AML LSC Populations

To confirm the in vivo observations, CD38−CD45RA+ cells and GMP-like populations were FACS-sorted from 5 AML patients (10 populations) and each population was cultured on MS5 stroma with cytokines (FIG. 6). The cultures were analyzed 4 and 8 days after culture initiation. After 4 days, most GMP-like cells remained CD38− and were CD90−CD45RA+ (FIG. 6B ii). The remaining input CD38− cells gained CD38 expression. By day 8, the original CD38−CD45RA+ cells were nearly all CD38+ and a proportion of cells has lost CD34 expression; consistent with differentiation into a CD34− blast population (FIG. 6B iii). The small numbers of CD34+CD38− cells were all CD90−CD45RA+, consistent with the immunophenotype of input cells.

After 4 days of culture of CD38+CD45RA+ cells nearly all cells remained CD38+ and some cells had already lost CD34 expression (FIG. 6B ii). There was little differentiation of CD34+CD38+ cells into CD34+CD38− cells.

Thus, the sum of the in vivo and in vitro data suggest that a CD38−CD45RA+ population lies at the top of hierarchy in most cases of primary human AML and differentiates into GMP-like population but notably both populations have LSC activity.

### Gene Expression Profiles of AML LSC and Normal Haemopoietic Stem/Progenitor Cells

We then obtained global mRNA expression profiles from 22 FACS-sorted AML CD38−CD45RA+, 21 GMP-like populations from 22 patients. In 18 patients we were able to obtain both CD38−CD45RA− and GMP-like AML populations allowing us to compare expression profiles between the two populations within each patient, thus negating the effect of genetic and epigenetic changes between patients. We also obtained from 5 normal HSC, MPP, CMP, GMP and CD38− CD45RA+ populations from 5 different age-matched human marrow samples. We asked two questions: first, are the two AML LSC populations (CD38− CD45RA+ and GMP-like) molecularly distinct; and second which normal populations are the two AML LSC populations most closely related to at a molecular level.

We used two approaches to determine if the expression profiles for the two AML LSC populations were distinct. First, we used a paired t-test (cut-off 0.01) to obtain a list of differentially expressed genes (917 probes; 748 mapped genes) between CD38−CD45RA+ and GMP-like cells from the subset of 18 AML cases where both populations were available from the same patient. The expression profiles of these differential genes was displayed by 3D Principal Component Analysis (PCA) (FIG. 7A). This shows that though the majority of the two populations are separated, 5/18 of the GMP-like populations lie interspersed with the CD38−CD45RA+ populations. This was confirmed standard t-test (cut-off 0.01) when applied to all AML population expression profiles—22 CD38− CD45RA+ and 21 GMP-like populations) was used to obtain a list of differentially expressed genes (458 probes; 360 genes) (FIG. 8A). We also examined the relationship between CD38−CD45RA+AML and GMP-like AML populations by hierarchical clustering with this minimum gene set (FIG. 8B). In this analysis, 5/21 GMP-like AML populations lie amongst CD38−CD45RA+AML populations and 1/22 CD38−CD45RA+AML population within the GMP-like AML populations.

Secondly, to obtain a more quantitative measure of the difference in gene expression between the two AML populations with LSC activity, we compared expression between CD38−CD45RA+ and GMP-like populations from the same patient, by a non-parametric (rank product) method (FIG. 7B). With a false discovery rate of 0.05 (pfp=0.5), 443 mapped genes were expressed more highly in CD38−CD45RA+ compared to GMP-like populations (FIG. 7Bi). Similarly, 1496 genes were more highly expressed in GMP-like compared to CD38− CD45RA+ populations (FIG. 7B ii). Using more stringent false discovery rate cut off of 0.01, 200 genes are expressed more highly in CD38−CD45RA+ populations and 943 genes are expressed more highly in GMP-like populations (data not shown). CD38 was amongst the top twenty most differentially expressed genes. Taken together, CD38−CD45RA+ and GMP-like LSC populations are molecularly distinct but show some overlap in gene expression and may not be fully separated.

To determine which normal populations the two AML LSC populations most closely resemble molecularly, we used ANOVA to curate a 2628 gene set (2789 probes) that maximally distinguished normal stem and progenitor populations. 3-D PCA was then used to display the profiles from the ANOVA curated gene set from normal populations (FIG. 7C). The signature of normal HSC was most closely related to the normal MPPs, with the normal CD38−CD45RA+, CMP and GMP populations more widely scattered. Each normal immunophenotypic population are relative closely co-located. Next, using this same 2628 ANOVA gene the expression profile of 22 CD38−CD45RA+ and 21 GMP-like populations AML populations were then distributed (FIG. 7Ci and ii). Both AML populations are more widely dispersed (presumably reflecting heterogeneity of genetic/epigenetic changes in AML). CD38−CD45RA+ and GMP-like AML populations mainly cluster around their normal counterpart population. However, some GMP-like AML populations are located closer to normal CD38−CD45RA+ cells.

Next, we used the same ANOVA gene set as a classifier to ask which normal population did each individual AML LSC populations most closely resemble (FIG. 7E-G). 17/22 (77%) CD38−CD45RA+AML populations were classified as normal CD38−CD45RA+ cells and 5/22 (28%) as GMPs. Only 278 probes (corresponding to 272 genes) (threshold 4.0) were required for the classifier. Additional genes did not provide further discriminatory power. Likewise for the GMP-like AML populations, 13/21 samples were called as GMP (62%), 7 as CD38−CD45RA+ (33%) and 1 sample as CMP (5%) using 241 probes (corresponding to 235 genes) (threshold 4.23). Similar results were obtained by hierarchical clustering using these same minimum gene sets that minimises misclassification error (thresholds of 4 and 4.23) (FIGS. 8C and D). 4/22 CD38− CD45RA+ AML populations were closer to normal GMP than normal CD38− CD45RA+ (FIG. 8C); whereas 5/21 GMP AMLs were closer to normal CD38− CD45RA+ than to normal GMP (FIG. 8D).

Taken together, the gene expression profiles of both AML populations with LSC activity do not map most closely to normal HSC.

Normal CD38−CD45RA+ Population has Cells with Lymphoid Primed Multipotential (LMPP) Potential.

The gene expression data above showed that global expression profiles of CD38−CD45RA+ AML most closely resemble normal CD38−CD45RA+ cells. The expression data suggested that the normal CD38−CD45RA+ population was distinct for other stem/progenitor cells. Previous studies had not shed light on the function of normal CD38−CD45RA+ cells. To investigate the lineage potential of CD38−CD45RA+ cells, we performed colony assays (FIG. 9A). HSC, MPP, CMP, GMP and MEP populations produced the expected lineage output. The CD38−CD45RA+ population had a cloning efficiency of −30% and produced granulocyte, macrophage and mixed granulocyte-macrophage colonies. Importantly, there was no erythroid output. When cells from the initial colony assay were replated, cells from CD38−CD45RA+ colonies had a replating potential intermediate between that of cells from MPP- and CMP-derived colonies (FIG. 5B). CD38−CD45RA+ cells exhibited no megakaryocyte potential, in contrast to MEP, CMP, HSC and MPP (FIG. 9C).

The B-lymphoid potential of CD38−CD45RA+ cells was tested by co-culture on MS5 stroma with cytokines. As positive control both HSC and MPP populations differentiated into myeloid (CD33-expressing) and B-lymphoid cells (CD19-expressing) cells whereas CMPs only gave rise to CD33+ cells (data not shown). CD38−CD45RA+ cells differentiated into both CD33+ and CD19+ cells (data not shown). The frequency of cells with myeloid, B- and T-lymphoid potential in the CD38−CD45RA+ population was determined by limiting dilution analysis (FIG. 9D-H). On MS5 stroma, 1/3.79 CD38−CD45RA+ cells had myeloid potential (compared to 1/12.4 GMP cells) (FIG. 9D). In conditions that promote both B-cell and myeloid output, 1/6.38 CD38−CD45RA+ cells differentiated into CD19 expressing B-cells and myeloid cells, whereas 1/95.79 GMP cells showed similar potential (FIGS. 9E and 9F). B-cell and myeloid potential was always seen together. Finally, the T-cell potential of the CD38−CD45RA+ cells was tested in an OP9-DL1 co-culture assay. 1/12 CD38−CD45RA+ cells expressed CD1a, CD7 and CD3 (FIGS. 9G and 9H) compared to 1/32 GMP cells. We also searched for early TCRd VD, DD or DJ and IgH DJ gene rearrangements in CD38− CD45RA+ cells, as compared to HSC, MPP, CMP and GMP controls but there was no evidence of significant polyclonal rearrangements at either loci in CD38− CD45RA+DNA or in control DNA (data not shown).

Expression of lymphoid- and GM-specific genes in CD38−CD45RA+ cells.

Murine multi-potential stem/progenitor cells express low levels of multiple lineage-affiliated gene expression programmes concordant with their lineage potentials (termed multilineage priming). As these cells pass through lineage restriction points, losing lineage potential, there is gradual, concomitant, extinction of lineage-affiliated gene expression programmes. In a refinement of this concept it has been suggested that there is a cascade of lineage-affiliated transcriptional signatures, initiated in HSCs and propagated in a differential manner in lineage-restricted progenitors (FIG. 10A).

Whether this also occurs in human haemopoietic stem/progenitor cells has not been previously reported. Therefore, we used quantitative RT-PCR to study expression of select lineage-affiliated genes shown in mouse to be representative of lineage-affiliated gene expression programmes, in 10 and 100 FACS sorted normal HSC, CD38−CD45RA+, GMP and MEP cells (FIG. 10B and FIG. 11). The aim was two-fold. First, to establish if lineage-affiliated patterns of gene expression seen in the mouse held true in human and, second, to determine if CD38−CD45RA+ cells expressed a lineage affiliated transcriptional programmes similar to murine LMPP cells (myelo-lymphoid gene expression and diminishing levels, or a lack of expression, of erythroid-megakaryocyte affiliated genes). The gene set chosen for study was based on previous published data and our own transcriptional profiling.

Consistent with previous data we found that in 10 FACS-sorted cells, MPL and HLF were most highly expressed in HSC (FIG. 10Bi) (representative of stem-cell only genes). Using our own gene expression data we confirmed that the stem/progenitor regulators BMI1 and MEIS1 were expressed in HSC and CD38− CD45R+ cells, with markedly lower expression in GMP and MEP. Similarly, KIT, IKZF1 (IKAROS) and RUNX1 are also expressed in HSC and CD38−CD45RA+ cells but also are expressed in GMP and MEP. Finally, HOXA9 and, to a lesser extent, IL3RA, are expressed principally only in CD38−CD45RA+ cells. Turning to the myeloid lineage affiliated genes, the early myeloid genes CEBPA, CSF3R, SPI1 (PU.1) are all expressed in HSC with expression retained in CD38− CD45RA+ and GMP cells but extinguished, as expected, in MEP (FIG. 10Bii). These are reminiscent of stem cell/myelolymphoid genes. The next pattern of myeloid gene expression is seen primarily in CD38−CD45RA+ and GMP cells (CSFR2A and GFI1) (i.e. restricted myelo-lymphoid) and, finally, the last layer of myeloid gene expression are late myeloid-specific genes (MPO and CSFR1), which are mainly expressed in GMPs only (differentiated myeloid). The pattern of early lymphoid gene expression is more focused; CD79A, ETS1, VPREB1, sterile IGHM, FLT3, NOTCH1 and RUNX3 all maximally expressed in CD38−CD45RA+ with little expression in other cell types (FIG. 10B iii). Finally, early stem-erythroid-megakaryocyte gene expression is epitomized by VWF, TAL1 and GATA2, which are all expressed in HSC and MEP. Both VWF and TAL1 are expressed at much lower levels in CD38−CD45RA+ cells, though GATA2 continues to be expressed in these cells, consistent with its broader role in haemopoietic progenitors (including GMP) biology. The erythroid-specific cytokine receptor EPOR, the erythroid-megakaryocyte transcription factor GATA1 and late erythroid transcription factor KLF1 show a more restricted pattern with expression principally in MEPs. Importantly, none of these three genes is expressed in CD38−CD45RA+ cells. Identical patterns of gene expression were obtained when either 10 or 100 FACS sorted cells where used (FIG. 10 and FIG. 11).

The sum of the gene expression data is consistent with multilineage priming in primary human stem/progenitors in a manner analogous to mouse. Moreover, these data are consistent with lymphoid and myeloid (GM), but lack of erythroid-megakaryocyte lineage potential of CD38−CD45RA+ cells described earlier. The sum of the functional and molecular data suggests that CD38−CD45RA+ cells are most similar to mouse LMPP cells.

### Example 2

### Prognostic Application of Diagnostic Screen of the Present Invention

A 49 year old male suffering from symptoms of pancytopenia presents himself to hospital. 10 ml of blood and/or 2 mls of bone marrow is removed for diagnostic. are for flow cytometery evlauation. The biological samples are treated either as in Example 1 or with red cell lysis buffer to remove red cells. Then the nucleated cells are incubated with antibodies as described in Example 1 that are either directly conjugated or indirectly conjugated. Excess unbound antibody is washed off. The stained cells are then put through a flow cytometer. Data is then collected and prognosis is made.

### Example 3

### Use of the Diagnostic Screen of the Present Invention in an in Vitro Assay to Identify a Therapeutic Candidate

A 33 year old with known Acute Myeloid Leukaemia present himself in hospital. 10 ml of blood and/or 2 mls of bone marrow is removed to monitor residual leukaemia stem cells for flow cytometery evlauation. The biological samples are treated either as in Example 1 or with red cell lysis buffer to remove red cells. Then the nucleated cells are incubated with antibodies as described in Example 1 that are either directly conjugated or indirectly conjugated. Excess unbound antibody is washed off. The stained cells are then put through a flow cytometer. Data is then collected and the effect of a therapeutic candidate assessed.

1. A diagnostic screen for detecting acute myeloid leukaemia, wherein said screen detects the presence (+) or absence (−), as indicated below, of the following cell surface polypeptide markers:
i) CD34+;
ii) CD45RA+; and
iii) CD90− and/or CD123+.

2. A diagnostic screen according to claim 1, wherein the marker iii) is CD90⁻.

3. A diagnostic screen according to claim 1, wherein the marker iii) is CD123⁺.

4. A diagnostic screen according to claim 1, wherein the marker iii) is CD90⁻ and CD123⁺.

5. A diagnostic screen according to claim 1, further comprising the cell surface polypeptide marker CD38+.

6. A diagnostic screen according to claim 1, further comprising the cell surface polypeptide marker CD38⁻.

7. A diagnostic screen according to claim 1, further comprising the cell surface polypeptide marker CD19⁻, CD47^{+/-}, CCR8^{+/-}, RHAMM^{+/-}, and/or CD 86⁻.

8. A diagnostic screen according to claim 1, comprising one or more antibodies that bind to one or more cell surface polypeptide markers selected from CD34, CD45RA, CD90, CD123, CD38, CD47, CD19, CCR8, RHAMM and/or CD86.

9. A diagnostic screen according to claim 8, comprising three antibodies, wherein:
a first antibody binds to CD34 and preferably not to CD45, CD90 and/or CD123;
a second antibody that binds to CD45RA and preferably not to CD34 CD90 and/or CD123; and
a third antibody that binds to CD90 and preferably not to CD34, CD45RA and/or CD123.

10. A diagnostic screen according to claim 8, comprising three antibodies, wherein:
a first antibody binds to CD34 and preferably not to CD45, CD90 and/or CD123;
a second antibody that binds to CD45RA and preferably not to CD34, CD90 and/or CD123; and
a third antibody that binds to CD123 and preferably not to CD34, CD45RA and/or CD90.

11. A diagnostic screen according to claim 1, for use in a method of diagnosis of acute myeloid leukaemia.

12. A method for diagnosis of acute myeloid leukaemia comprising:
i) contacting an isolated sample containing a blood cell population with a screen according to claim 1;
ii) confirming the presence of a blood cell that has a cell surface phenotype comprising:
a) CD34⁺;
b) CD45RA⁺; and
c) CD90⁻ and/or CD123⁺.

13. A diagnostic screen according to claim 1, for use in a method of prognosis of acute myeloid leukaemia.

14. A method for prognosis of acute myeloid leukaemia comprising:
i) contacting an isolated sample containing a blood cell population with a screen according to claim 1;
ii) confirming the presence of a blood cell that has a cell surface phenotype comprising:
a) CD34⁺;
b) CD45RA⁺; and
c) CD90⁻ and/or CD123⁺.

15. A diagnostic screen according to claim 1, for use in a method of identifying a therapeutic candidate for the treatment of acute myeloid leukaemia.

16. A method of identifying a therapeutic candidate for the treatment of acute myeloid leukaemia comprising:
i) contacting the therapeutic candidate with an isolated sample containing a population of blood cells, wherein said blood cell has a cell surface phenotype comprising:
a) CD34⁺;
b) CD45RA⁺; and
c) CD90⁻ and/or CD123⁺;
ii) incubating said therapeutic candidate with said isolated sample;
iii) contacting said isolated sample after step ii) with a screen according to claim 1;
iv) identifying blood cells by step iii) that have a cell surface phenotype comprising:
a) CD34⁺;
b) CD45RA⁺; and
c) CD90⁻ and/or CD123⁺;
v) correlating the number of blood cells identified by step iv) with the number of blood cells present in an isolated sample prior to step i) that have a cell surface phenotype comprising:
a) CD34⁺;
b) CD45RA⁺; and
c) CD90⁻ and/or CD123⁺;
vi) confirming the presence of a therapeutic candidate having anti-acute myeloid leukaemia cell activity by identifying a relative decrease in the number of blood cells in step v) after contact with the therapeutic candidate; or
confirming the absence of a therapeutic candidate having anti-acute myeloid leukaemia cell activity by identifying no significant relative decrease in the number of blood cells in step v) after contact with the therapeutic candidate.

17. A diagnostic screen according to claim 1, for use in a method of monitoring efficacy of a therapeutic molecule in treating acute myeloid leukaemia.

18. A method of monitoring efficacy of a therapeutic molecule in treating acute myeloid leukaemia comprising:
i) contacting an isolated sample from a patient, wherein said patient has been administered the therapeutic molecule, with a screen according to claim 1;
ii) identifying blood cells by step i) that have a cell surface phenotype comprising:
a) CD34⁺;
b) CD45RA⁺; and
c) CD90⁻ and/or CD123⁺;
iii) correlating the number of blood cells identified by step ii) with the number of blood cells present in an isolated sample taken from a patient prior to administration of the therapeutic molecule, wherein said blood cells taken prior to administration of the therapeutic molecule have a cell surface phenotype comprising:
a) CD34⁺;
b) CD45RA⁺; and
c) CD90⁻ and/or CD123⁺;
iv) confirming efficacy of the therapeutic molecule by identifying a relative decrease in the number of blood cells in step iii) after contact with the therapeutic molecule; or
confirming the absence of efficacy of the therapeutic molecule by identifying a no significant relative decrease in the number of blood cells in step iii) after contact with the therapeutic molecule.

19. A kit for diagnosis and/or prognosis of acute myeloid leukaemia, said kit comprising at least one antibody that binds to one or more cell surface polypeptide markers selected from:
i) CD34;
ii) CD45RA; and
iii) CD90 and/or CD123; and optionally
iv) CD38, CD47, CD19, CCR8, RHAMM and/or CD86.

20. (canceled)
